(19) United States
(12) Patent Application Publication
(10) Pub. No.: US 2022/0277254 A1
(43) Pub. Date: Sep. 1, 2022

(54) CONTEXTUALIZED SENSOR SYSTEMS

(71) Applicant: Aptima, Inc., Woburn, MA (US)

(72) Inventors: John Feeney, Beavercreek, OH (US); Kevin Durkee, Carmel, IN (US); Zachary Kiehl, Dayton, OH (US); William Depriest, Dayton, OH (US); Matthew Ewer, Fairborn, OH (US)

(73) Assignee: Aptima, Inc., Woburn, MA (US)

(21) Appl. No.: 16/728,972

(22) Filed: Dec. 27, 2019

Related U.S. Application Data
(60) Provisional application No. 62/785,615, filed on Dec. 27, 2018.

(51) Int. Cl. G06Q 10/06; G08B 21/02; G08B 25/10; H04W 4/38

(52) U.S. Cl. CPC G06Q 10/063114; G08B 21/02; G08B 25/10; H04W 4/38

(57) ABSTRACT

A contextualized sensor system is provided comprising one or more sensors, one or more memory elements, a library of alert rules stored in the one or more memory elements, one or more processors, and the one or more memory elements including instructions that, when executed, cause the one or more processors to perform operations comprising: receiving from one of the one or more sensors one or more sensor data, comparing the first sensor data to a library of alert rules to determine whether an alert situation has occurred, and communicating an alert if the alert situation has occurred. In some embodiments, the operations further comprise contextualizing an environmental data, a location data, a physiological data, a behavior data and an orientation data.

### CROSS-REFERENCE TO RELATED APPLICATIONS

This application claims the benefit of U.S. App. No. 62/785,615, filed on Dec. 27, 2018, entitled “CONTEXTUALIZED SENSOR SYSTEMS,” the entire contents of which is incorporated herein by reference.

### STATEMENT REGARDING FEDERALLY SPONSORED RESEARCH OR DEVELOPMENT

This invention was made with Government support under Contract Nos. FA8501-16-C-0020 and FA8501-16-C-0027 awarded by the U.S. Air Force. The Government has certain rights in the invention.

### REFERENCE TO SEQUENCE LISTING, A TABLE, OR A COMPUTER PROGRAM LISTING COMPACT DISC APPENDIX

Not Applicable.

### BACKGROUND OF THE INVENTION

### 1. Field of the Invention

This invention relates to objectively monitoring working environments in unusual spaces, in particular systems to contextualize sensor data related to humans working in unusual situations such as confined spaces.

### 2. Description of the Prior Art

In many cases when trying to use unobtrusive wearable sensors to trigger health and safety assurance alerts from workers in unconventional postures, there was no objective way to disambiguate a normal state from an abnormal state. Commercial off-the-shelf (COTS) wearable sensors, while commonly available, often have limitations to their effectiveness when being worn by workers in unconventional positions. The standard body locations for wearable sensors for monitoring activities such as exercising or doing normal daily tasks are often not suitable for working in unconventional positions.

Common wearable sensors face two significant issues when used to monitor workers. The first issue is that they cannot be repositioned to a more acceptable location for workers in unconventional positions. These wearable sensors are designed for mass appeal with standard locations, such as the wrist. Many of these sensors are incapable of being worn in alternative locations. The second issue with COTS wearable sensors concerns the data they provide. The sensors are designed to collect a specific type of data for monitoring of the wearers (e.g., heart rate) and not for advanced analytics or alerting. These sensors typically look to generate this specific data over periods of time to determine a generalize description of the wearer's activities. For example, a heart rate monitor computes the wearer's heart rate to estimate the amount of time the wearer is in particular heart rate zone or to assess what level of activity they were performing. In this way, a typical heart rate monitor is designed to monitor and report activity levels, not to alert the user of issues, because they are not designed or capable of including data from other sensors.

Additionally, most health and safety alerts (a.k.a. alarms) are based on static thresholds with no regard to individual differences (i.e., idiosyncrasy) or context (e.g., activity of the user). Physiological alerts, such as those triggered by high or low heart rates, typically use a one-size-fits-all model without accounting for differences in baseline physiology or current activities. This shortcoming typically leads to an abundance of false health and safety alerts, which ultimately contributes to alert fatigue and increased overall health and safety risks.

A need exists to address the shortcomings currently in this field.

### BRIEF SUMMARY OF THE INVENTION

The following summary is included only to introduce some concepts discussed in the Detailed Description below. This summary is not comprehensive and is not intended to delineate the scope of protectable subject matter, which is set forth by the claims presented at the end.

In one example embodiment, a contextualized sensor system is provided comprising one or more sensors, one or more memory elements, a library of alert rules stored in the one or more memory elements, one or more processors and the one or more memory elements including instructions that, when executed, cause the one or more processors to perform operations comprising: receiving from one of the one or more sensors one or more sensor data, comparing the first sensor data to the library of alert rules to determine whether an alert situation has occurred, and if the alert situation has occurred, communicating an alert.

In some embodiments, the one or more sensors comprises an environmental sensor, a location sensor, a physiological sensor, a behavior sensor and a posture sensor, and the one or more sensor data comprises an environmental data, a location data, a physiological data, a behavior data and a posture data.

In some embodiments, a processor based contextualized sensor system is provided comprising a body area subsystem comprising one or more sensors configured to collect state data of a worker; a monitoring subsystem comprising: an expert subsystem comprising a situation classifier module and an intervention module, a subsystem database comprising contextualization rules and a library of alert rules, the situation classifier module is configured to determine a contextualized situation from one or more sensor data using the contextualization rules, and the intervention module configured to compare the contextualized situation to the alerting library to determine whether an alert situation has occurred; and if the alert situation has occurred, the monitoring subsystem configured to communicate an alert.

In some embodiments, the situation classifier module further comprises a state contextualizer module configured to determine a contextualized state of the worker from the one or more sensor data. In some embodiments, the contextualized state is determined from an orientation data. In some embodiments, the contextualized state is determined from a location data. In some embodiments, the contextualized state is determined from a comparison of the one or more sensor data to a baseline data of the one or more sensor.

In some embodiments, the one or more sensor data comprises an environmental data, a location data, a physiological data, a behavior data and a posture data. In some embodiments, the one or more sensors comprise an environmental sensor, a location sensor, a physiological sensor, a behavior sensor and a posture sensor.

In some embodiments, a processor based contextualized sensor system for determining a contextualized state of a worker is provided, the system comprising a body area subsystem comprising one or more sensors configured to collect state data of a worker; and a monitoring subsystem comprising: an expert subsystem comprising a situation classifier module, a subsystem database comprising contextualization rules, the situation classifier module is configured to determine a contextualized state of the worker from the state data.

In some embodiments, the contextualization rules comprise a raw variable, a contextualization variable and a resulting contextualized variable, the raw variable comprises a first raw state data of the worker, the contextualization variable comprises a second raw state data of the worker and the contextualized variable comprises an objective representation of the contextualized state of the worker given the first raw state data and the second raw state data of the worker. In some embodiments, the first raw state data comprises a heart rate of the worker. In some embodiments, the second raw state data comprises a comparison metric of a core body temperature of the worker to a baseline core body temperature of the worker. In some embodiments, the second raw state data comprises an orientation data of the worker.

In some embodiments, the body area subsystem further comprises one or more environmental sensors configured to collect environmental data and the situation classifier module is further configured to determine a contextualized state of the worker from the state data and the environmental data. In some embodiments, the contextualization rules comprise a raw variable, a contextualization variable and a resulting contextualized variable, the raw variable comprises a raw state data of the worker, the contextualization variable comprises an environmental data, and the contextualized variable comprises an objective representation of the contextualized state of the worker given the raw state data and the environmental data. In some embodiments, the state data comprises a heart rate of the worker. In some embodiments, the state data comprises a comparison metric of a core body temperature of the worker to a baseline core body temperature of the worker. In some embodiments, the environmental data comprises a location data of the worker. In some embodiments, the environmental data comprises a carbon monoxide level.

In some embodiments, the operations further comprise contextualizing one or more of the environmental data, the location data, the physiological data, the behavior data and the posture data.

### BRIEF DESCRIPTION OF THE SEVERAL VIEWS OF THE DRAWINGS

In order that the manner in which the above-recited and other advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1A shows a process diagram illustrating the general concepts of one example embodiment of a Contextualized Sensor System (CSS);

FIG. 1B shows a process diagram illustrating a system overview of one example embodiment of a CSS;

FIG. 1C shows a functional diagram illustrating an architecture of one example embodiment of a CSS;

FIG. 1D shows a functional diagram of one example embodiment of components of a CSS;

FIG. 2A shows a functional diagram illustrating an architecture of one example embodiment of a Body Area Subsystem (BAS);

FIG. 2B shows a functional diagram illustrating an architecture of one example embodiment of a status band;

FIG. 3A shows a function diagram illustrating an architecture of one example embodiment of a situation classifier module;

FIG. 3B shows a function diagram illustrating an architecture of one example embodiment of a decision support module and an intervention/alert module;

FIG. 3C shows a function diagram illustrating an architecture of one example embodiment of web applications;

FIG. 4A shows an example embodiment of a body worn sensor of a BAS;

FIG. 4B shows an architecture overview of one example embodiment of a location sensor;

FIG. 4C shows an example embodiment of a status band;

FIG. 5 illustrates one example embodiment of a computer system suitable for a contextualized sensor system; and

FIGS. 6A-6C show different embodiments of CSS components.

### DETAILED DESCRIPTION OF THE INVENTION

COPYRIGHT NOTICE: A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. The following notice applies to any software and data as described below and in the drawings hereto: Copyright © Aptima, Inc., 2018-2019, All Rights Reserved.

Contextualized sensor systems for unconventional environments and methods of use will now be described in detail with reference to the accompanying drawings. It will be appreciated that, while the following description focuses on a system that provides a contextualized sensor system for confined spaces, the systems and methods disclosed herein have wide applicability. For example, the contextualized sensor systems described herein may be readily employed in other situations such as but not limited to mammals in space or underwater environments where traditional sensor data may not accurately reflect the state of the mammal. Notwithstanding the specific example embodiments set forth below, all such variations and modifications that would be envisioned by one of ordinary skill in the art are intended to fall within the scope of this disclosure.

As used herein, the term “module” refers to hardware and/or software implementing entities and does not include a human being. The operations performed by the “module” are operations performed by the respective hardware and/or software implementations, e.g. operations that transform data representative of real things from one state to another state, and these operations do not include mental operations performed by a human being.

The terms “sensor data”, as used herein, is a broad term and is to be given its ordinary and customary meaning to a person of ordinary skill in the art (and are not to be limited to a special or customized meaning), and furthermore refers without limitation to any data associated with a sensor, such as an electrocardiogram (ECG) monitor, inertial measurement unit (IMU), force sensing resistors (FSR), electromyography (EMG) monitors, thermometer, hygrometer, goniometer, a camera device, a heart rate monitor, an accelerometer, a photodetector and a reflectance-based pulse rate monitor.

The term confined space, as used herein, is a broad term and is to be given its ordinary and customary meaning to a person of ordinary skill in the art and furthermore refers without limitation to areas that are considered “confined spaces” because while they are not necessarily designed for people, they are large enough for workers to enter and perform certain jobs. A confined space may also have limited or restricted means for entry or exit and is not designed for continuous occupancy. Confined spaces include, but are not limited to, tanks, vessels, silos, storage bins, hoppers, vaults, pits, manholes, tunnels, equipment housings, ductwork, pipelines, etc. A confined space may also have two or more of the following characteristics: contains or has the potential to contain a hazardous atmosphere; contains material that has the potential to engulf an entrant; has walls that converge inward or floors that slope downward and taper into a smaller area which could trap or asphyxiate an entrant; or contains any other recognized safety or health hazard, such as unguarded machinery, exposed live wires, or heat stress.

### The Technical Problem

One technical problem this solution addresses is how to automatically recognize unsafe work conditions to improve work safety in unconventional working environments. This challenge presented by this problem is increased when the recognition of unsafe environments has to be done from a remote location.

For example, in one particular environment, aircraft maintenance work is a mission-critical function posing various potential hazards to human performers that must be accounted for through health and safety monitoring practices. Among the most important areas to consider in this maintenance work are confined spaces, such as fuel tanks, dry bays, tunnels, and landing gear pods. These confined spaces can be small in size, contain adverse atmospheric conditions, and are often located in areas that are not visible or easily accessible to an outside observer. Workers that enter confined spaces may encounter a number of potentially serious hazards including insufficient oxygen supply, flammable or explosive atmospheres, toxic gases, and electrical or mechanical hazards. Hence, safety assurance practices must be performed by a standby attendant for the entire duration a worker is inside a confined space, including voice communication at frequent and pre-specified intervals to monitor the person's status. To meet these safety requirements, significant personnel time and resources are needed to man the standby attendant roles during the performance of any work inside of confined spaces; often at a 1-to-1 ratio of standby attendants per every worker in a confined space. Since the focus of each standby attendant is solely on maintaining voice communication at specific intervals while monitoring for problems, they are unable to perform any other functions. Furthermore, the communication intervals are often spaced out in increments, such as at 15-minute increments, meaning there could be a delay in the identification of an emergency situation and response time if a person in a confined space was injured between communication times. This increases the risk of severe injury or loss of life.

Other remote monitoring attempts to provide effective alerting revolves around the mitigation of false health and safety alerts, which in this context includes an alert of a potential issue when there is no issue actually present. In the current context, threats to workers in unconventional environments, which may force users to be in unconventional postures, coupled with the serious consequences of failing to detect an issue require a system with high sensitivity. This high sensitivity—or the high probability of detecting a dangerous state for the workers—leads to issues with generating too many false alerts, thus indicating there is a health or safety issue with the worker when in actuality there is not.

The issue of a system with a high number of false alerts or low specificity makes any attempt at a preventative alerting system ultimately futile. There are many examples where systems with high false alerts rates lead to users either (1) ignoring the alerts or (2) simply turning the alerting off if possible. As alluded to previously, we refer to this observation as alert or alert fatigue.

The problem requires a system with the ability to address three main challenge: the first challenge is the ability to capture data from the worker in the unconventional orientations or postures which raises the issues of the size, location, and capabilities of the sensors; the second challenge is how to determine if the worker is in a normal or a compromised state based on current data or trends observed in the collected data; and the third challenge is how to provide accurate and effective alerting when users are in a compromised state or trending towards a compromised state.

### The Technical Solution

The technical solution to address the above technical problems is generally to create and utilize an optimized real-time alerting engine based on the fusion of sensor and contextual data. This solution addresses two system requirements: high sensitivity and high specificity. In other words, the approach includes a system that has the capacity to detect adverse states of a worker while producing very few false alerts.

Embodiments of the technical solution provide: (1) personal and environmental sensors collecting data from the worker and their environment, (2) location tracking sensors that are not reliant on GPS; (3) contextualization algorithms and tables for assessment of maintainer health and safety based on sensor data; (4) a decision support station that enables safety attendants to readily monitor maintainer health and safety while identifying hazards with low false alert occurrences; and (5) support capabilities for proactively identifying and responding to intervention needs, including planning and executing effective courses of action (COAs) for emergency response collaboration.

The challenge of having the ability to measure the worker in the unconventional orientations or postures is addressed through the development and deployment of custom and COTS sensors to provide estimates of the workers' statuses within their respective environments.

The challenge of remotely determining if the worker is in a normal or a compromised state is addressed through processing and fusion of the data sensors in a way that allows them to establish a snapshot of the worker and provide an associated contextualized state of a worker (e.g., laying prone in a specific location which is a confined space).

The challenge of providing accurate and effective alerting when users are in a compromised state or trending towards a compromised state is addressed through expert-derived data-driven rules to determine if an alert is warranted for a particular set of conditions.

For the solution, it is not expected that a single sensor will have the sensitivity to disambiguate when a worker in an unconventional posture and physical space is in a physiologically normal or a compromised state. Because of this challenge, the fusion of multiple streams of sensor and contextual data is able to provide the data necessary to determine an accurate estimate of the worker state.

To assist with this objective, multiple sources of data may be used, such as but not limited to: (1) location data, (2) environmental data, (3) physiological data, (4) behavioral data, and (5) physical orientation (e.g., posture) data. While there are intersections between these data sources (by design), each data source provides insight into the underlying situation or condition.

The sensors serve as initial inputs to the system, as additional processing and fusion is performed before a determination of worker state, posture, or activity can be ascertained.

Generally, the sensors provide raw data that is processed to produce measures of interest for use within the subsequent steps of the process. After the sensor data is collected and processed, measures are computed. These measures are then fused with each other to align them both temporally and by location. The temporal fusion process ensures measures are combined concurrently and in the correct temporal order. Likewise, the fusion of location data ensures that measures and sensors are collocated in the same space. For example, physiological and motion data can be fused with atmospheric data based on the location data of the worker.

Once fused, the measures are then used in two distinct functionalities. The first function of these measures is to serve as an input to predictive models to provide early-warning alerts. These predictive models focus on the prediction of trends in key data sources and measures, such as cardiovascular data and heart rate. The predicted values and additional rule-based logic are used to generate an alert to indicate the potential for an adverse worker state. The second function of the measures is to assist in real-time monitoring and alerting. This function is achieved by the utilization of a library of clinically derived alerting rules that were developed based on a team of medical clinicians. More specifically, this alerting library focuses on describing specific physiological conditions that could be assessed using the data available.

In summary, embodiments of the described solution use a fusion of disparate data sources to create a more accurate system for health status alerting within confined spaces or environments otherwise not meant for human habitation.

### Differences from Prior Solutions

The disclosed contextualized sensor system integrate multiple sources of sensor data (e.g., location, environmental, physiological, behavioral, and orientation/posture data) to provide context to a human health and safety sensing system which is different from many prior art solutions when the specific use case of health and safety monitoring is within a confined spaces. There are very few health and safety monitoring systems that focus on typical work activities that comprise confined space work. Out of the few systems that do exist, the inventors are unaware of any that use sensor fusion and contextual data as a method to mitigate the number of false positive alerts. Other existing systems may exist incorporate one or two data sources; however, they typically assume normative conditions during their sampling. For example, typical use of a COTS heart rate monitor assumes normal postures and atmospheric conditions. While these assumptions are acceptable for many applications, they are not acceptable within a capricious and potentially dangerous environment, such as that of an aircraft fuel tank.

Another difference from prior art is that the discussed system accounts for differences in baseline physiology. As mentioned previously, there is a fair amount of between-subject variability in baseline physiology and behavior. Additionally, there is often even day-to-day variability for the same individual. This observation is accounted for in the current system design by the collection of baseline data at the beginning of each worker's shift, which is ultimately used to calculate individualized alerting thresholds.

In summary, the fusion of sensor data, contextual information, and worker baseline information allows for the development of a hierarchical alerting paradigm that is intelligent and adaptable. Though some of these features may exist in other systems, the exist in isolation to the best of our knowledge. Because of these two reasons, we believe the proposed system is substantially different from prior art, especially when the specific application of confined space monitoring is incorporated.

### A Practical Application

The contextualized sensor system recognizes and solves the technical problems related to monitoring humans working in unusual situations.

The resulting product enables real-time sensing of maintainers and their surrounding environments as they operate in confined spaces and other potentially hazardous areas. CSS supports prevention, detection, and intervention of health and safety hazards while greatly reducing the time, costs, and manpower required by current confined space monitoring practices. This solution has an objective of providing capabilities to report, view, and control all factory operations and resources across different facilities, and to enhance depot productivity through reduced machine and process downtime. Compared to current-day operations that require safety attendants at a one-to-one ratio, CSS allows a many-to-one ratio of maintainers per safety attendant, thereby reducing costs and increasing efficiencies. The CSS may provide an enterprise-wide solution that reduces costs, improves performance, and increases reliability across all weapon systems.

For example, the disparate data sources described above are combined to be important indicators of worker state in unconventional postures. The utilization of five sources of data and subject matter expert (SME) recommendations is used to derive alerting rules to produce non-intuitive results. The derived rules are not simple syllogism of individual sensor data, but rather a complex integration of the different data sources, which ultimately results in fewer false alerts and increased specificity. The derived rules account for a complex interplay between the actions of the workers, their expected physiological response to these activities, and the impact of the environmental conditions they find themselves in.

Additionally, the resulting physiological data of workers in unconventional orientation/postures doing unconventional activities can differ from conventional postures and activities, such as sitting or standing. Changes in physical posture can skew certain physiological signals (e.g. digit-derived photoplethysmogram). For example, compression of the diaphragm may result in lower respiration rates or respiratory tidal volume.

Without the integration of data such as posture, position, and activity, the resulting suppression in respiration could result in increased false alerts. Likewise, the combination of unconventional postures and activities could lead to an increased heart rate, thus erroneously indicating a state of distress.

By integrating the data sources and understanding the normal underlying physiological responses of workers in unconventional postures doing physically exerting tasks, it is possible to assess worker state more accurately.

This solution also uniquely incorporates the features of new sensors and communication protocols. Prior to the miniaturization of requisite components and availability of short-range wireless communication protocols, real-time multi-modal data acquisition and transition simply would not have been possible.

While more examples could be provided, it is sufficient to say that the fusion of disparate data streams to provide contextualized alerts is not an obvious solution for application within dangerous work environments such as OSHA-defined confined spaces.

### One Embodiment of the Contextualized Sensor System:

The described contextualized sensor systems for context-enabled multi-sensor fusion for optimized health and safety alerting during unconventional physical postures is comprised of a system with numerous components. At a high level, the system uses an architecture for routing the requisite data along with a context-enabled hierarchical alert paradigm. Within the architecture, there are numerous subcomponents, such as sensors for different data sources, software modules for processing the data, and devices for relaying both data and health status alerts.

### System Goals

Some of the goals of the CSS include: supporting multiple operationally relevant roles charged with ensuring the health and safety of individuals operating in confined spaces; providing adequate, clear, and role-appropriate situational awareness regarding confined spaces, entrants, and roving attendants in monitored areas; providing detection and actionable alerting of states and events that pose a potential risk to the health and safety of individuals operating in confined spaces; providing a real-time monitoring station software that enables a person to perform the roles and responsibilities of a remote attendant from a remote location; providing capabilities that enable a person to perform the role and responsibilities of a roving attendant on an as needed/floating basis; providing support for emergency responders to be notified and provided with actionable information in response to a person's request for emergency intervention; providing clear and distinct real-time physiological information about individuals operating in confined spaces; providing clear and distinct real-time atmospheric sensing information within the confined spaces just prior to and during the entire time individual(s) are operating within those spaces; providing clear and distinct real-time awareness of which individual(s) are currently in each confined space; providing clear real-time awareness of the locations of roving attendants working in the monitored areas; providing a means for a confined space entrant to communicate their intent and status clearly and simply to the appropriate person(s); being able to detect and alert in real-time potential risks to health and safety using the sensor data available to the system; being able to detect and alert in real-time potential risks to health and safety due to inadequate or faulty sensor data; being able to detect and alert in real-time potential risks to health and safety due to loss of connectivity between system components; and being able to detect and alert in real-time when individual(s) are in confined spaces without access approval or otherwise required to exit the space.

### Risk Detection

In operation, the CSS may be configured to detect and notify relevant person(s) of detectable conditions that potentially pose a threat to the health and safety of individuals operating in confined spaces. For example, the CSS may detect relevant atmospheric hazards (e.g., abnormal 02, LEL, JP-8 levels) and alert the remote attendant, it may detect relevant health issues (e.g., abnormal cardiorespiratory) and alert the remote attendant. The CSS may also employ alerting models to indicate system component failures (e.g., sensor disconnects, network failure).

### User Roles

The CSS supports the operationally relevant roles for safely operating in confined spaces. In addition to the user, these roles may include a remote attendant and a roving attendant.

For a role of the remote attendant, the CSS provides a real-time monitoring station software that enables a person to perform the roles and responsibilities of a remote (standby) attendant from a remote location. The CSS provides remote attendants the ability to view geographical locations of all active entrants on a map display. The CSS provides remote attendants the ability to view lists of all active entrants on a single view. The CSS provides remote attendants the ability to select a specific entrant and monitor health and safety indicators for that individual. The CSS provides remote attendants the ability to select a specific entrant and monitor atmospheric safety indicators for that individual. The CSS provides remote attendants the ability to receive and view alerts (e.g., emergency alert (red alert); early warning (yellow alert); pending entry request (blue alert); system failure (orange alert)). The CSS provides remote attendants the ability to approve/deny pending entry requests for signature by formal supervisor. The CSS provides remote attendants the ability to see when an entrant has exited or checked out of a space. The CSS provides remote attendants the ability to indicate within the system an emergency situation has occurred.

For the role of the roving attendant, the CSS provides capabilities that enable a person to perform the role and responsibilities of a roving (standby) attendant on an as needed/floating basis. The CSS provides roving attendants the ability to receive alerts (i.e., emergency alert (e.g., red alert); early warning (e.g., yellow alert)). The CSS provides roving attendants the ability to initiate and/or confirm problematic events.

### System Overview

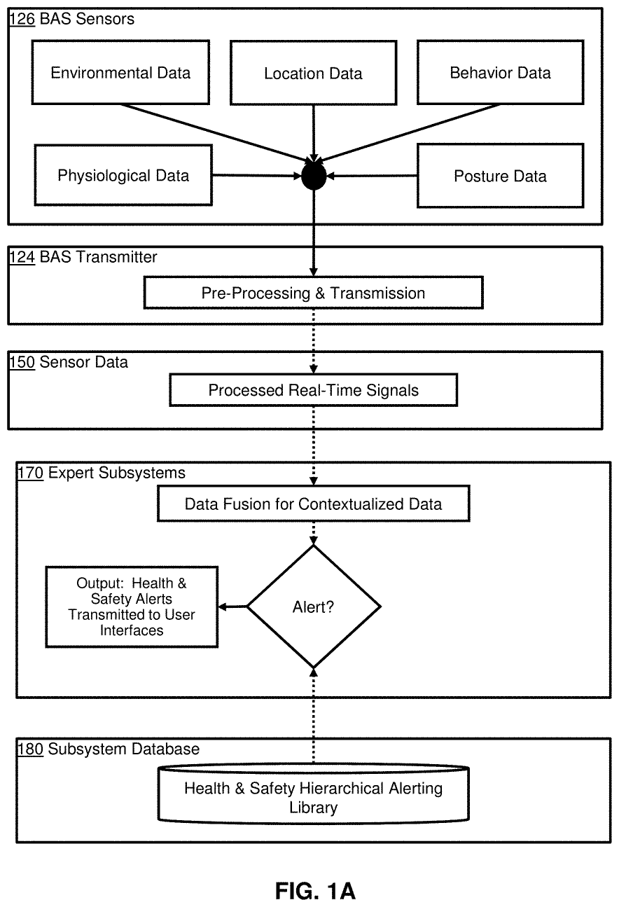

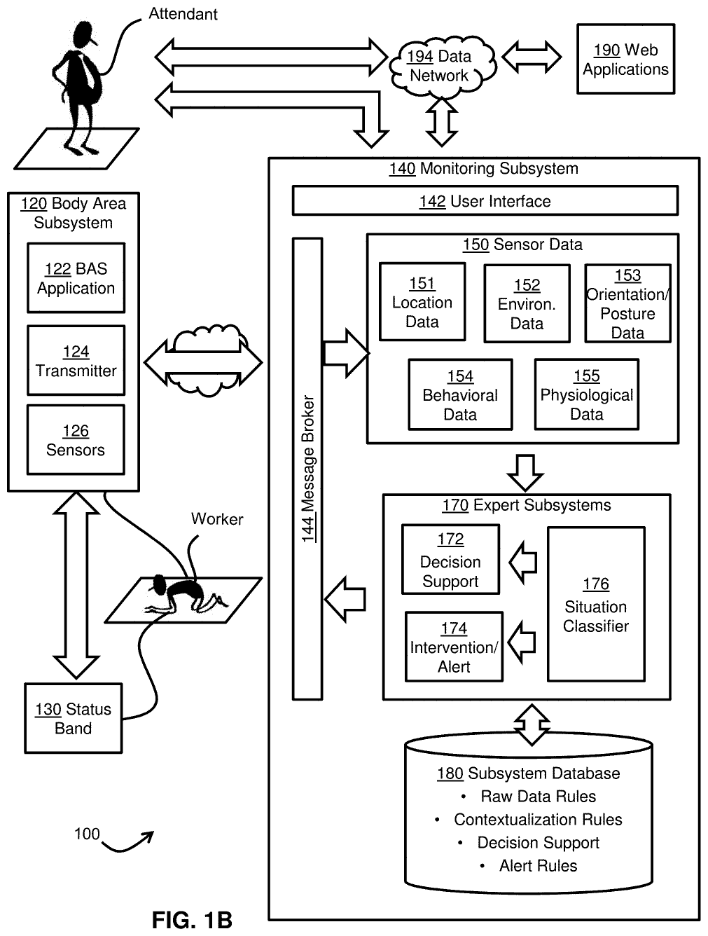

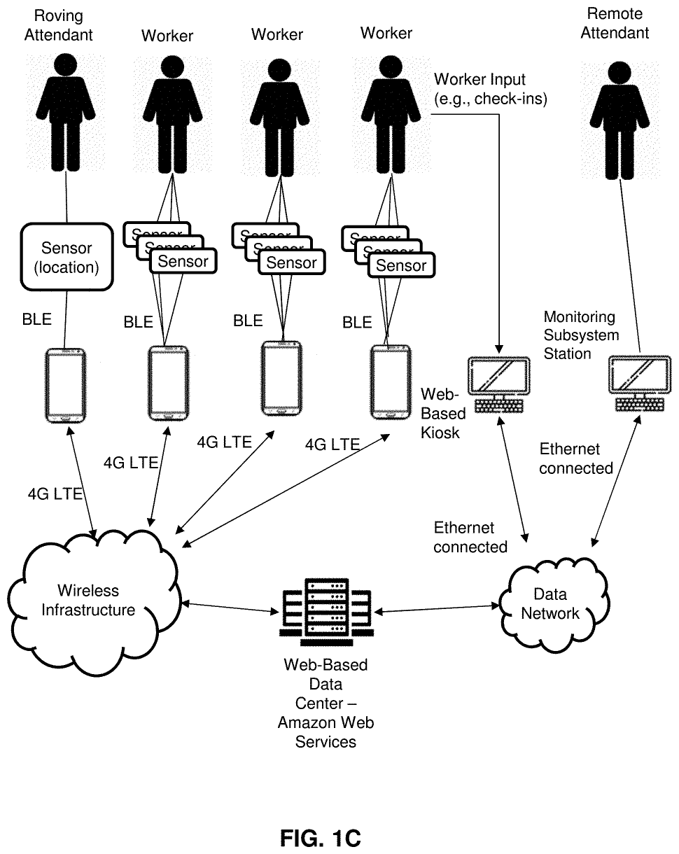

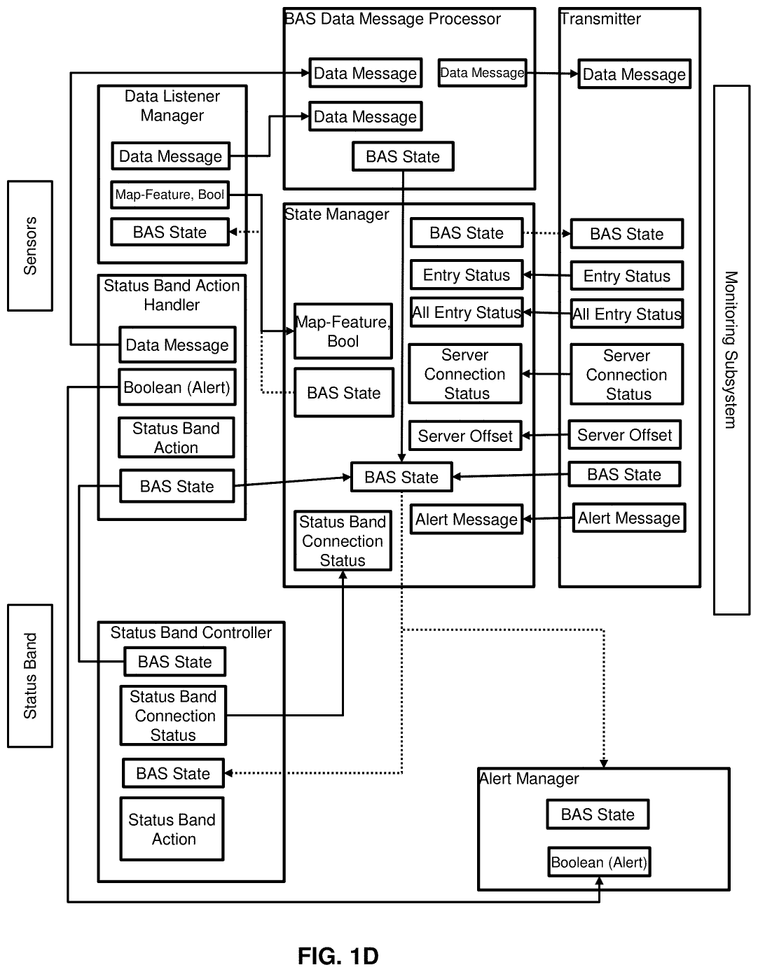

The general architecture of one example embodiment of the CSS is shown in FIG. 1B. As shown, the CSS 100 comprises a body area subsystem 120, a status band 130 and a monitoring subsystem 140. The body area subsystem 120 generates and transmits data sources from the worker and the monitoring subsystem 140 uses this data to classify the state of the worker and provides decision support or interventions based on that state. The status band 130 is typically on the work and provides an interface from the monitoring subsystem 140 to the worker and may include special alerting interfaces. Optionally, the CSS 100 may further comprise web applications 190 that allow the CSS 100 to communication information to remote users.

In embodiments, the personal or body area subsystem 120 may comprise sensors 126, a transmitter 124 and BAS applications 122. The sensors 126 generally capture data local to the worker, the transmitter 124 communicates this data to the monitoring subsystem 140 and the BAS application 122 provides configuration and other features to the BAS 120. The transmitter 124 also receives data from the monitoring subsystem 140 such as alerts and status data.

In embodiments, the status band 130 generally provides a local status to the worker. The status band 130 may communicate alerts to the worker and may allow the worker to provide additional data to the monitoring subsystem 140.

In embodiments, the monitoring subsystem 140, also called the CSS Server, generally enables the CSS 100 to share information between all other components, save data, and preserve system state.

In embodiments, the web client applications 190 generally allow for storage of data and allow for remote status and remote management of the CSS 100, CSS subsystems, algorithms, and data.

Operationally, referring to FIG. 1A, the activity flow of the CSS generally comprises receiving sensor data from BAS sensors 126, pre-processing and transmitting that with the BAS transmitter 124 to the monitoring subsystem as processed sensor data 150 for data interpretation and fusion, determining contextualized states by the expert subsystems 170. The expert subsystems 170 will take the fused data together with alerting data from the system database 180 to determine whether an alert state exists. If an alert state exists, an alert will be communicated to the BAS and/or status band and/or attendance interface. In some embodiments, the CSS may communicate system information to web clients for remote monitoring of the data.

FIG. 1C shows the architecture design may utilize Bluetooth Low Energy (BLE), cellular/wireless communications infrastructure, and web/cloud-based services using Infrastructure as a Service (IaaS).

FIG. 1D shows details of an example embodiment of a CSS with some of the CSS, BAS and status band components showing the flow of data among these components.

### Body Area Subsystem (BAS)

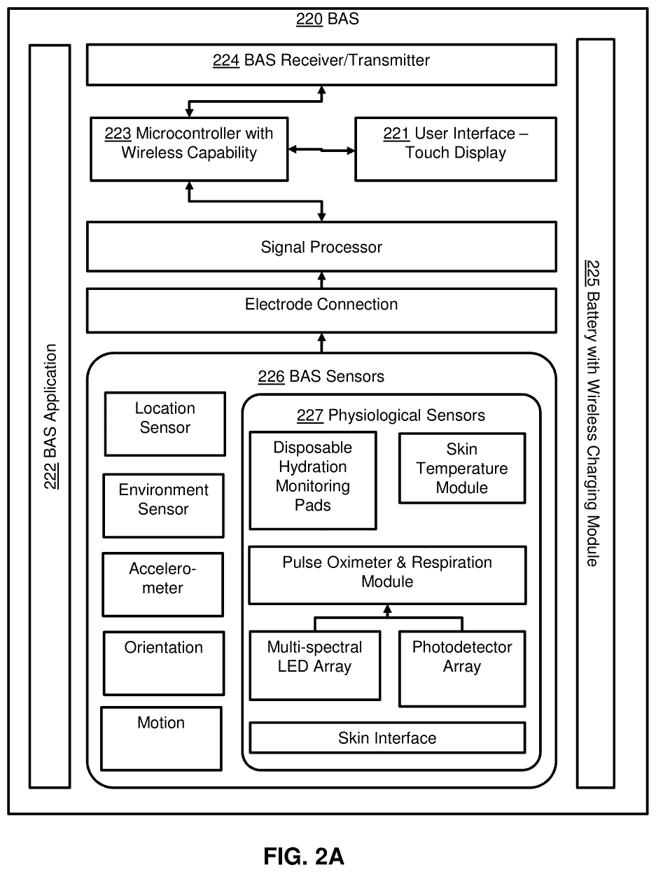

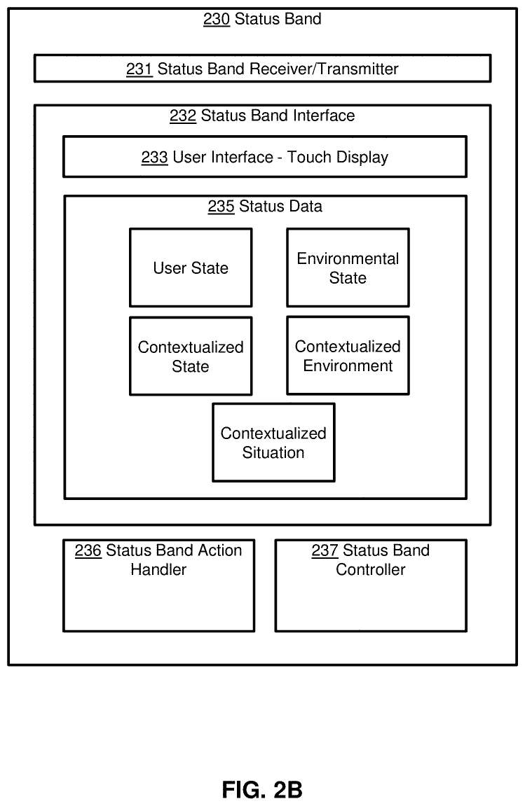

Referring to FIG. 2A, The BAS 240 generally serves as a gateway or intermediary between the CSS server, the status band and the BAS sensors 226. The BAS 220 may also provide additional contextualization information for the system. The BAS 220 generally comprises a BAS application 222, BAS sensors 226 and a BAS receiver/transmitter 224.

The BAS 220 bridges each maintainer's sensor data to the CSS cloud server by being installed on common devices such as an Android phone, connecting to each sensor via BLE, performing the necessary real-time processing, and transmitting the resulting data features to the server via the 4G LTE capabilities on each phone. Secure websockets are then used to immediately push out data updates from the CSS server to the decision support station. Furthermore, in some embodiments, the BAS 220 may perform some CSS functions locally to effectively load balancing all this data contextualization, because if you did everything on the CSS server it would be prone to overloading and lack scalability.

Referring to FIG. 2A, the BAS application 222 may comprise several components. For example, the BAS application 222 may comprise a synchronization module to synchronize the data from the BAS sensors. Another module, the filtering module, handles intelligent data filtering/signal processing routines (basically any data transformations), to manage RF bandwidth (i.e., sending certain data at higher/lower granularity compared to other data, based on how best to contextualize) and a prompting module to prompt the person being sensed/monitored for responses at certain times to further improve the contextualization.

The BAS application 222 may also be configured to allow authorized users to configure various settings with the application, such as which sensors to connect to. These settings allow the application to be customized to specific deployment environments without having to build specialized applications. This application requires users to first be authenticated via a login page and only permits users of certain roles to be authorized to use the app and make changes. This provides a layer of security to prevent an unauthorized user from inadvertently (or maliciously) making changes to the application.

The BAS transmitter/microcontroller 224 may comprise smartphones, smartwatches, and short-range communication protocols such as BLE; mobile cellular infrastructures capable of long-term evolution (LTE) telecommunication; (3) a cloud computing platform for housing data management, algorithms, and health status alerts; and (4) a central monitoring station for monitoring the status of workers in unconventional postures and locations.

The BAS sensors 226 may comprise any type of sensor that can provide information regarding a worker or the working environment. These sensors may be physiological sensors, environmental sensors, behavioral sensors or positional sensors. For example, in one embodiment, the sensors comprise skin temperature sensors, pulse oximetry sensors, accelerometers and location sensors.

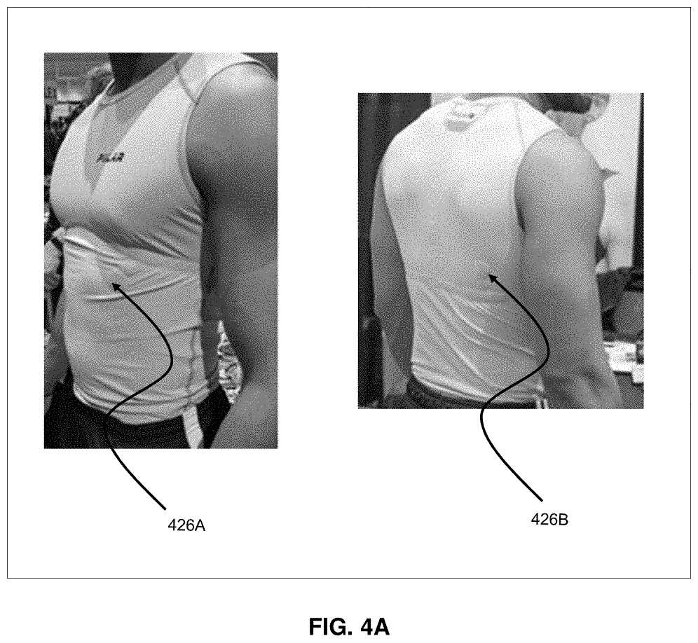

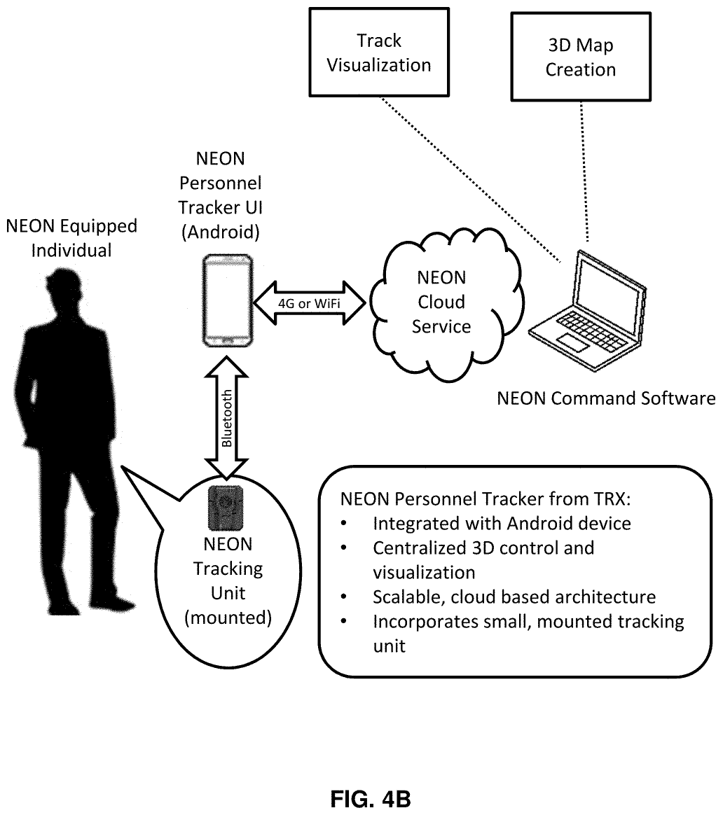

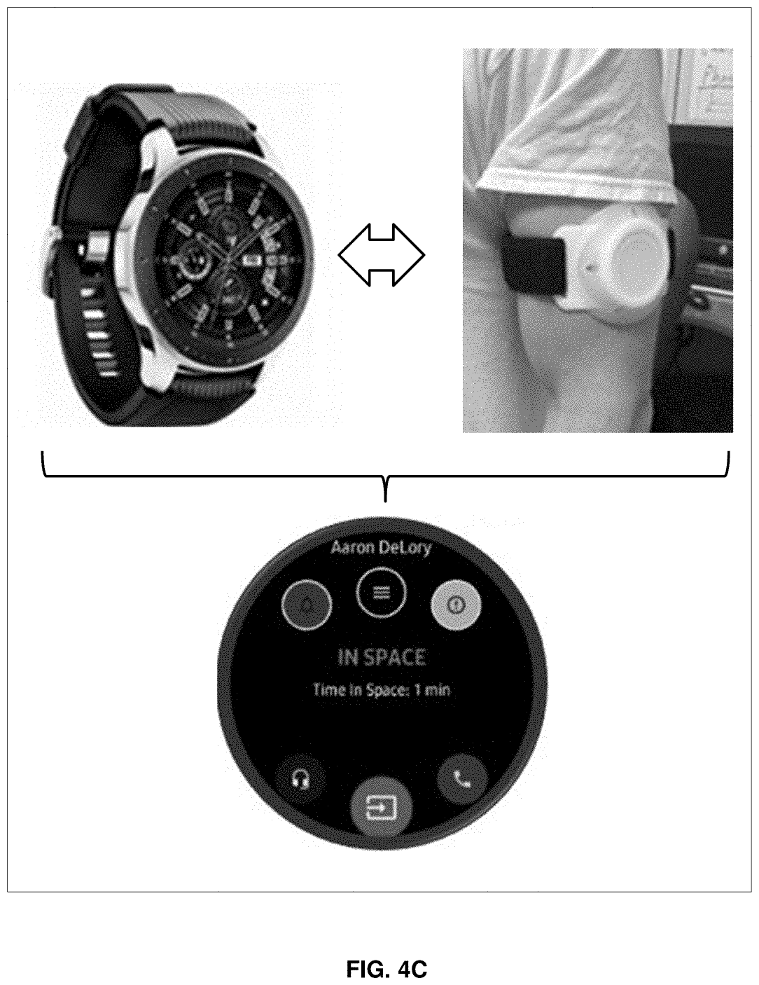

FIG. 4A shows an example embodiment of a body warn BAS showing physiological sensors 426A and 426B under the garment and against the worker's skin.

### Body Area Subsystem (BAS) Receiver/Transmitter

The BAS receiver/transmitter 224 generally provides the communications link between the BAS and the monitoring subsystem. In some embodiments, the BAS receiver/transmitter 224 is configured with wireless connectivity to provide the ability to connect and live stream each maintainer's sensor data to the monitoring subsystem elements such as decision support station displays. This prevents the server from having to manage sensor connections directly, making the CSS more scalable. Additionally, because the BAS can remotely connect to the server (via WiFi or cellular network), users do not need to stay within a certain physical range of the server (making them more mobile). Another benefit of the BAS receiver/transmitter is that it runs as a background service. This ensures the application is always running and maintaining an active connection with the server. It also limits user interaction with the system, which allows users to focus on their tasking without distraction.

The BAS receiver/transmitter 224 may comprise smartphones, smartwatches, and short-range communication protocols such as BLE; mobile cellular infrastructures capable of long-term evolution (LTE) telecommunication; (3) a cloud computing platform for housing data management, algorithms, and health status alerts; and (4) a central monitoring station for monitoring the status of workers in unconventional postures and locations.

### Body Area Subsystem (BAS) Sensors and Data

The CSS uses physical objects—such as COTS sensors and sensor components—as input devices (i.e., sensors to provide data display mechanisms) and output devices (e.g., display mechanisms). The solution may incorporate existing physical objects; however, it also uses these objects to produce numerous new features. One example is the use of a smartwatch, which uses reflectance-based pulse rate monitors and an onboard accelerometer to provide estimated heartrate and motion. The smartwatch also serves as a multi-modal alerting/display mechanism by providing auditory, visual, and haptic alerts to the human worker if dangerous conditions or activities are detected.

Referring to FIG. 2A, the BAS sensors 226 are generally selected to provide data such as, but not limited to: (1) location data, (location in comparison to a known layout); (2) environmental data; (3) physiological data; (4) behavioral data, and (5) physical data such as body orientation or posture data.

Location data supports the identification and tracking of workers while in the working area. This allows for the development of derived metrics, such as establishing both worker location and length of time within one location.

Environmental data is used to describe ecological conditions immediately surrounding the worker (i.e., an assessment of the ambient environment). Key sources of environmental data include temperature, humidity, and both the presence and concentration of atmospheric gases in the general vicinity. The types of atmospheric data include (1) standard atmospheric gases such as Oxygen, Carbon Dioxide, Hydrogen and (2) hazardous gases such as Methane, Nitrogen Dioxide, Sulfur Dioxide, Hydrogen Sulphide, Carbon Monoxide, Carbon Dioxides, and Volatile Organic Compounds (VOCs).

Physiological data is used to help determine the physiological state of the worker. In an illustrative embodiment, physiological data is used to monitor the worker's cardiopulmonary system. To achieve this objective, a number of physiological data sources are used, such as cardiovascular data, pulmonary data, and heat stress data. Each source of physiological data provides insights into specific conditions of the workers. For example, cardiovascular data assists in determining if a worker's heart is functioning as expected (i.e., is their heart rate within a normal range?). Additionally, cardiovascular information can be used to determine core body temperature, which is a leading indicator for heat-related illnesses. Pulmonary data assists in determining if the worker is breathing normally, such as the number of breaths per minute.

Behavioral data may be used to provide context on level of activity, types and pattern of motion and activity, and tasks being performed by the worker.

Physical orientation or posture data may also be used to assist in describing the worker's motion, position, and posture. This posture data is generally based on an orientation of the worker to a baseline orientation. For example, if an IMU is baselined for a standing position of the worker, the IMU can sense when the worker is 90 degrees from standing and this can be assumed to be a prone position.

One example of suitable wearable technology is the combination of a wearable gas sensor and smartwatch for carbon monoxide (CO) monitoring. These two sensors may be used to provide data that can be fused with location data, posture data (e.g., laying on side), behavioral data (e.g., no movement in the last 30 seconds), and environmental data (e.g., high CO levels) to (1) monitor the environment, (2) monitor the individual's health, and (3) alert the individual through various means if they are in danger.

The CSS may maintain role-appropriate situational awareness regarding confined spaces, entrants, and roving attendants in the monitored areas.

The CSS may be capable of monitoring the locations of individuals both in geodetic coordinates and relative to the confined spaces. The CSS location tracking system may be configured to: track the geodetic location of roving attendants while they are outside of any confined spaces; track the geodetic location of approved entrants while they are outside of any confined spaces; detect the entry of an individual into a confined space; and detect the exit of an individual from a confined space.

Location data may be provided by both embedded (i.e., within other devices and components) and stand-alone components. The specific components and associated data sources include micro-electro-mechanical system (MEMS) such as accelerometers or inertial measurement units (IMUs), which house additional components like gyroscopes and magnetometers. Other components that can provide location data include global positioning system (GPS) transmitters and Bluetooth Low Energy (BLE) or ultra-wideband (UWB) beacons and transmitters. While some of these components are housed within standalone casing, many of them are subcomponents to larger wearable/transportable devices (e.g., smartphones, smartwatches).

The CSS may be capable of monitoring key environmental atmospheric indicators as required for maintaining awareness of health and safety of entrants to the confined spaces. Environmental atmospheric sensors may be capable of monitoring: O2 percentage; LEL percentage; and Broadband VOCs (e.g., JP-8 levels).

The CSS may be capable of monitoring key physiological indicators as required for maintaining awareness of health and safety of entrants to the confined spaces. CSS physiological sensors may be capable of monitoring: Heart rate; Respiratory rate; and Motion rate (via accelerometry).

In one example embodiment, components for physiological data collection may comprise an electrode-embedded garment and chest strap. Together, these two components can collect data sources such as an electrocardiogram, time between sequential heart beats (often referred to as R-R or inter-beat intervals), respiration rate and waveform, and skin temperature. These components also allow for the derivation of other important physiological metrics, such as tidal volume, core body temperature, and heart rate variability.

In one example embodiment, components for environmental data collection consist of hand-held atmospheric monitors, wearable atmospheric monitors, and embedded temperature and humidity sensors.

In one example embodiment, components of the BAS that provide behavior data largely overlap with the components of the posture data and the location data. Sensors such as accelerometers and photodetectors provide context for the type of the activity the human is doing along with the level of activity. For example, the fusion of these data sources allows remote monitoring personnel to determine if someone is using a power tool in a dark space. Again, most of these sensors are embedded within other wearable devices.

Lastly, posture/orientation data largely relies on data provided by components such as MEMS/IMU. After a fair amount of digital signal processing, raw movement and posture data can be fused together to create an estimate of the human operator's posture.

Another set of CSS components are those interfaces that display the status of humans during unconventional work tasks, postures, and locations. There are three separate points where this display happens: (1) at the level of the individual worker via smartwatch display, (2) at the level of a roving attendant via internet-enabled tablet, and (3) at the level of a remote attendant via a traditional desktop PC and monitors. The health status alerts, which are derived from the aforementioned data sources and sensing components, can then be transmitted to each of the involved parties.

In many embodiments, a non-GPS location tracking capability is needed to provide real-time positional data on each maintainer within an indoor maintenance complex. CSS benefits from reliable and accurate maintainer location tracking capabilities that conform to the constraints of aircraft maintenance environments, particularly ALCs. The main challenge in meeting this goal is that most commercial location tracking systems are GPS-based, and connections to a GPS constellation are unavailable from inside a space like an aircraft hangar.

To address this challenge, a solution based predominantly on microelectromechanical systems (MEMS) was used. MEMS is based on precise sensing of inertial navigation to determine the location of an individual within a defined area. The major advantage of MEMS is that location is determined primarily by the wearable sensor itself (i.e., locally). MEMS technology is also very energy-efficient, which supports the need for extensive daily usage without having to re-charge batteries. Another consideration is that permit-required confined spaces may contain remnants of flammable gases or dust, requiring all sensors entering permit-required spaces to be intrinsically safe. Several factors in designing intrinsically safe electronics devices include: reducing/eliminating internal sparking, controlling component temperatures, and eliminating component spacing that would allow dust to short a circuit. MEMS location sensor technology is capable of being intrinsically safe by operating with low voltage. This helps pre-position CSS for eventual use in permit-required confined spaces.

One challenge of MEMS is the potential for sensor drift, which requires occasional re-calibration to properly correct. Frequent calibration incurs excessive interruptions with maintainers using the technology. To address this challenge, calibration would be accelerated and streamlined, or auto-calibration followed immediately by wirelessly transmitting location data from its wearer to a CSS connection point. After location data are routed to CSS, the information would later be integrated into the decision support station's display, providing graphical depictions of where all maintainers are in respect to confined space entrances.

In an example embodiment, MEMS technology provided by TRX Systems, Inc. named the NEON Personnel Tracker was used. As shown in the overview of FIG. 4B, the NEON sensor fusion and mapping software uses information from low-cost MEMS sensors (gyroscope, accelerometer, etc.) to deliver location and context in indoor, underground, and other environments without GPS. TRX produces a small, wearable sensor accessory, the NEON Tracking Unit, and NEON Indoor Location software to enable ubiquitous mapping and location indoors. The MEMS sensors on board the NEON Tracking Unit estimate relative location of the wearer, then apply constraints based on installed beacons within the work environment and/or map information. This blended solution allows for high location tracking accuracy and with fewer beacon placements than other competing solutions. The use of multiple fused data sources also allows NEON to auto-calibrate and initialize location tracking with minimal time and effort by end users. The NEON wearable sensor communicates via BLE with a mobile device running the Android OS, which then distributes the location data to third-party applications subscribed to its Application Programming Interface (API). The NEON location service provides enhanced location accuracy by communicating with the NEON cloud service to reference beacon locations and pre-mapping data, at which point the MEMS data from the sensor unit is fused with these additional sources. For the CSS application, the BAS is configured to connect directly to the TRX NEON API to receive real-time location data for each individual. The BAS application facilitates sending location data to the CSS server so it can be processed and viewed by remote safety attendants. Extensive testing of the NEON platform was performed in several different environments, with various beacon types (including BLE options), with differing beacon densities, and from both inside and outside of a confined space. When properly configured, NEON performed adequately well in virtually all test cases mentioned, typically obtaining less than 10 feet of error (frequently much better, e.g., <5 feet), which is ample accuracy to rapidly locate an individual under distress.

### Status Band

The status band generally provides an on-person contextualization aid. Referring to FIG. 2B, the status band comprises a receiver/transmitter 231, a status band interface 232, a status band action handler 236 and a status band controller 237.

The status band 230 provides notifications to workers and roving attendants (RAs) via the BAS, including: (1) their current state in the system, (2) connection statuses with the server and sensors, and (3) relevant alerts. It also provides a way for workers to enter or exit a confined space, and when appropriate, to request assistance and/or call for help. This is accomplished by interfacing with the BAS. The BAS relays information it gets from the server to the status band. The status band is currently built as Tizen web application and can be deployed to Samsung smartwatches.

The status band 230 is designed to communicate directly with the BAS. The status band receiver/transmitter 231 communicates information from the BAS, which includes status data 235. The status band interface 232 may be a touch display 233 and indicators for status data 235. Status data 235 may comprise representations for status such as user state, contextualized state, contextualized situation, environmental state and contextualized environment. The status band 230 may also sending data to the BAS (and eventually the CSS server) with the receiver/transmitter 231.

In some embodiments, data from the status band 230 is communicated via the Bluetooth protocol using single connection.

There are two types of data the status band 230 communicates with, one is for receiving status updates from the BAS through the BanStatusWrapper, and the other is sent to the BAS via the InteractionMessage.

In some embodiments, the status band 230 receives a status message every 5 seconds from the BAS, which serves as a heartbeat mechanism and to ensure the two components' statuses are in-sync. Status data are also sent to the status band on demand whenever changes to the BAS status occur. If the status band 230 fails to receive a status message within 10 seconds, it will notify the user that it is disconnected from the BAS. Requests sent out from the status band are sent to the BAS and relayed to the server from there. These messages must be sent and received by the server within 3 seconds for them to be fully processed.

Communicating over Bluetooth within the status band is accomplished via the Samsung Accessory Protocol (SAP).

During initialization, the status band creates callbacks using this API to determine if the connection to the BAS is successful, if it failed, or if it became disconnected. It also creates a callback to appropriately handle data that is received from the connection. Once these callbacks are initialized, an attempt to establish a connection is initiated and handled accordingly.

### Monitoring Subsystem (CSS Server)

The monitoring subsystem, also referred to as the CSS server, enables the system to share information between all other components, save data, preserves system state and provides the core contextualization engine for the CSS. The monitoring subsystem is generally responsible for managing the data within the CSS. This includes collecting and transferring data from locally worn devices to a server which stores and processes the data. It is also responsible for disseminating data to other components, including sensor data, entry state changes, and any alerts derived from the processing.

To serve information to multiple clients simultaneously, the monitoring subsystem is based on a client-server architecture. Having a centralized system server allows the system to be managed in one place, which makes administrative tasks such as data backup or exporting much easier. There are also pre-existing tools and documentation available to enable fast and efficient development of a server using this architecture.

The CSS leverages a cloud-based infrastructure such as Amazon Web Services (AWS) to deploy the monitoring subsystem. Using a cloud-based infrastructure limits the amount of physical hardware that needs to be purchased and maintained to run the server. Another benefit of AWS is there is support for the GovCloud data center, which has already been approved for government use.

Referring to FIG. 1B, the monitoring subsystem generally comprises a user interface 142, a message broker 144, sensor data 150, expert subsystems 170 and a subsystem database 180.

The user interface 142 provides an interface for users, such as an attendant, of the monitoring subsystem 140.

The message broker 144 allows the monitoring subsystem 140 to stream large amounts of data in and out in real-time to many different components. In one example embodiment, ActiveMQ was chosen to serve as the CSS's message broker 144 because the tool provided all the necessary functionality needed by the system including being able to reliably transfer data and provide multiple transport methods (such as AMQP and WebSockets) to communicate with the other components in the system.

The sensor data 150 is provided from the BAS 120 which may include (1) location data 151, (2) environmental data 152, (3) physiological data 155, (4) behavioral data 154, and (5) physical orientation/posture data 153.

This raw data 150 is fed to the expert subsystems 170 that fuse, or contextualize, the data to determine the contextualized state, the contextualized environment and in some embodiments, the contextualized situation. The expert subsystem 170 may comprise a series of pre-defined rules defined based on a long series of information synthesis from literature, interviewing subject matter experts, and research findings in data collected. The rules may be stored in the subsystem database 180 and allow for a determination of contextualized worker states such as the worker being in a compromised state or trending towards a compromised state. Similar rules for the environment may allow for a determination of a contextualized environment such as high levels of CO which may be acceptable in one space but not acceptable in another. Together, the contextualized worker state and contextualized environmental state may provide a contextualized situation.

Furthermore, the CSS may comprise decision support modules 172 that provide decision support to the worker and CSS user. This decision support modules 172 may enable safety attendants to readily monitor maintainer/worker health and safety while identifying hazards with low false alert occurrences.

The CSS may further comprise further support capabilities from an intervention/alert module 174 for proactively identifying and responding to intervention needs, including planning and executing effective courses of action (COAs) for emergency response collaboration.

The subsystem database 180 contains data used by the monitoring subsystem and the expert systems. For example, the subsystem database may store rules and thresholds to define alert measures, contextualization tables to help define contextualizes states and environments based on raw sensor data, decision support data to be use to provide additional information for decisions given a contextualized data and a library of alert rules from which particular alerts may be selected based on the contextualized data.

### Expert Subsystems—Situation Classifier

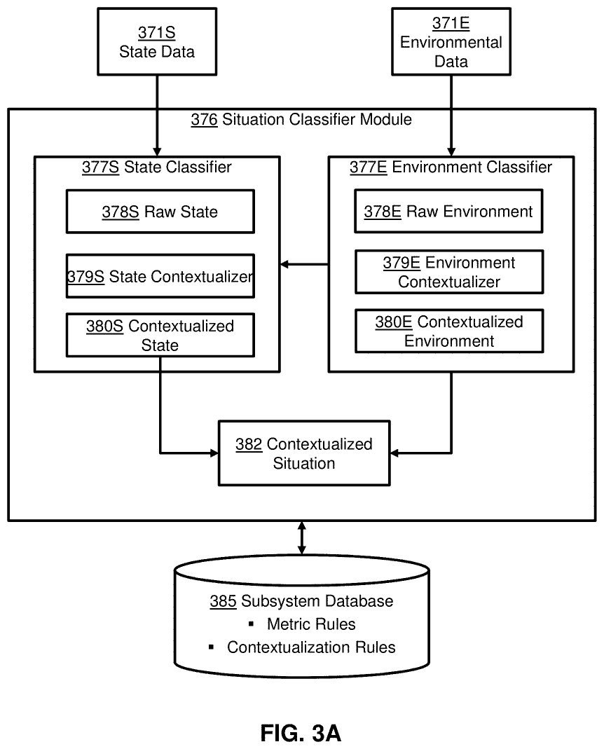

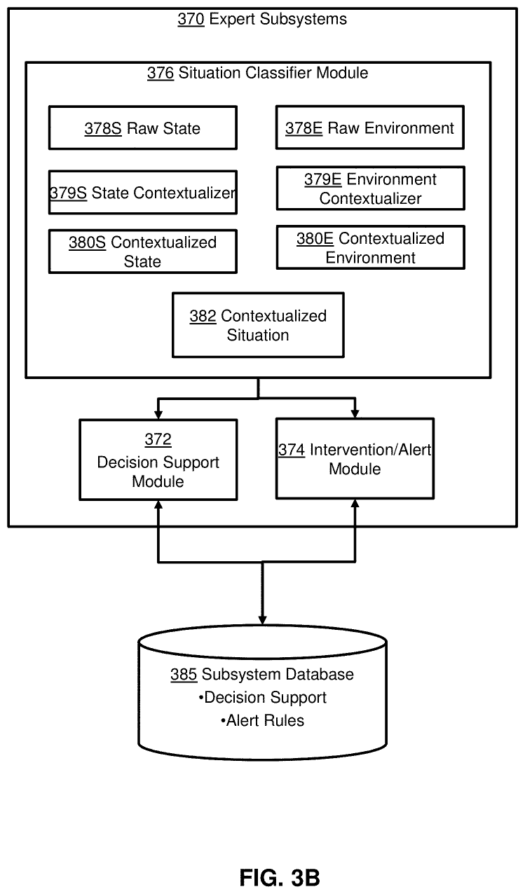

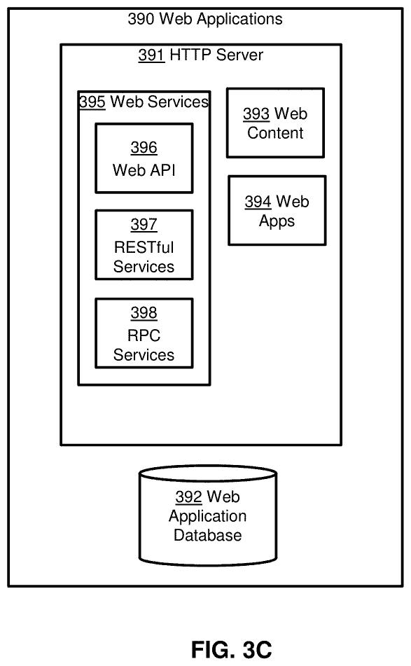

FIG. 3A illustrates an example embodiment of the expert subsystems' situation classifier module 376 in more detail. As shown, raw data from the worker and environmental sensors comprising state data 371S and environmental data 371E is received and communicated to the situation classifier module 376. The situation classifier module 376 generally performs the contextualization of the state and environmental data given the raw data. Within the situation classifier module 376, raw data from the sensors is communicated to a state classifier module 377S and an environmental classifier module 377E. Within these classifier modules, the raw data may be used alone or the data may be fused with other data to create metrics reflecting a raw state for the worker and a raw environment for the environment. The raw data is then used by contextualizer modules to contextualize the raw data and define the contextualized state 380S and the contextualized environment 380E. Together, the contextualized state and environment may be used to define a contextualized situation 382.

For example, within the state classifier module 377S, raw worker state data 378S is received as state data 371S and may include raw data such as EKG data, body temperature, location data or IMU orientation. This raw worker state data 378S may also be fused with other information to create raw state metrics. This fusion and metric computation may be done independently prior to contextualization according to a series of predefined metric rules stored in the subsystem database 385. Computed metrics may include metrics such as but not limited to Heart-Rate (HR) metrics, breathing metrics, body motion metrics, body temperature metrics, body orientation/posture metrics or comparison metrics of data to a baseline data. HR metrics may be derived from EKG data and may include metrics such as but not limited to rapid HR acceleration, HR cessation or unnatural heart rate changes/variability. Breathing metrics can be derived from R-R intervals of EKG data or respiratory inductance plethysmography and may include metrics such as but not limited to a breathing cessation metric. Body motion metrics may be derived from location data, accelerometer data, or a combination of location and accelerometer data and may include metrics such as but not limited to motion acceleration or cessation metrics. A body temperature metric may be derived from a temperature sensor and these metrics may include metrics such as but not limited to a core body temperature metric defined by a baseline core body temperature fused with HR metrics to compute core body temperatures over time and a sustained high core body temperature metric. A body orientation/posture metric may be derived from calibrated IMUs and may include metrics such a but not limited to an orientation of the sensor as compared to a pre-calibrated orientation baseline and these metrics may define the orientation of the worker as being in a prone or standing position. This raw data and metrics may be used to define the raw state of the worker.

Similar to the processes in the state classifier module 377S, environmental data 371E from environmental sensors may be used alone or with metrics to determine the raw state of the environment within the environment classifier module 377E. Raw environmental data may include data from environmental sensors such as but not limited to data reflecting CO concentration, location data or ambient temperature.

This raw state data 378S and raw environment data 378E may then be contextualized to determine a contextualized state with the state contextualizer module 380S and the environmental contextualizer module 379E.

Contextualization of raw state and raw environment data is generally performed according to a series of predefined rules stored in the subsystem database 385. The rules may define relationships between variables such as a raw variable, a contextualization variable and a resulting contextualized variable. The raw data variables may include the raw data and metrics as described above. Contextualization variables may include any other data that provides contextual information for the raw data variable. The contextualization variable may comprise other raw data or other contextualized data. Contextualized variables comprise a pre-defined objective interpretation or representation of the state of the worker or the environment given the raw data variable and the contextualization variable.

Generally, the matching of raw data variables and contextualization variables can be matched to contextualized variables according to contextualization rules in the subsystem database 387.

To illustrate an embodiment of contextualizing raw state data 378S, the raw state data variables may comprise (1) a Heart-Rate (HR) metric from an EKG sensor and (2) a motion metric calculated from a location sensor. Raw environment data may comprise (1) a CO level near the worker and (2) a metric reflecting the CO levels rising over a period of time. In this example, the contextualization variable data may comprise the CO level of the worker as data to provide context to the motion metric of the worker. In this case, if the raw data variable is a motion metric reflecting no motion of the worker, and the CO level is used as a contextualization variable, a predefined rule for the contextualized variable may reflect a determination of a contextualized state of the worker state being unconscious due to high CO levels. In contrast, if the motion metric (raw state variable) reflects motion of the worker yet the CO level as the contextualization variable gets to a level that exceeds a pre-defined threshold, the contextualized variable of the worker may reflect the worker being conscious but it may reflect the worker as approaching a compromised state due to the high CO levels.

As environmental data 371E was used as the contextualization variable for the raw state variables of the work in the example above, other raw state data, other raw environmental data or any combination of this data may be used as the contextualization variable to define the contextualized state given the raw data. The contextualization rules in the subsystem database 385 define these rules and the relationship between the raw variables, the contextualization variables and the contextualized variables. In some embodiments, the relationship is defined by a lookup table being pre-populated with the relationships of the variables. In some embodiments, additional modeling techniques such as machine learning (ANNs, SVMs, Deep Learning) and Bayesian models are alternate implementations that could be used to learn or more accurately define the relationships between the raw data variables and the contextualized variables.

In some embodiments, the contextualized state and environment may be objectively determined from algorithms that use the raw data variables or contextualized variables to calculate additional variables. For example, health status classifier algorithms for assessment of maintainer health and safety may be determined from a combination of raw data variable values based on pre-determined mathematical relationships.

In some embodiments, the contextualized state 380S and the contextualized environment 380E are used by a contextualize situation module 382 according to contextualization rules in the subsystem database 385. The contextualized situation represents a combined context of the contextualized state 380S and the contextualized environment 380E.

### Expert Subsystem—Decision Support and Intervention/Alert

With the contextualized state, environment and situational data, the CSS monitoring subsystem includes a processing module that executes algorithms and executes multiple rules based on derived features within the system to evaluate and prioritize contextualized states to generate alerts. For example, multiple states may be evaluated at any given time so that the prioritization of alerts ensures maintainer health and safety.

Referring to FIG. 3B the expert subsystem 370 may further comprise a decision support module 372 and an intervention/alert module 374. As shown, these modules may take raw or contextualized data from the situation classifier module 376 and use this data to identify decision support or intervention/alert information. Decision support or intervention/alert information may be obtained using tools similar to how raw data is contextualized. For example, the decision support or intervention/alert information may be pulled from a lookup table in the subsystem database 385 given the raw or contextualized data. Decision support or intervention/alert information may also be obtained by algorithms that mathematically determine decision support or intervention/alert situations. Decision support or intervention/alert information may also be enhanced by weighting different variables based on the importance of that variable to the situation.

The decision support module 372 provides users with improved decision making by providing consistent and optimal solutions for situations encountered. The decision support module 372 may provide novice users with the knowledge and support to make decisions at the level of a subject matter expert. For example and not for limitation, decision support information may be provided to address data that reflects a trend of variables towards an alert situation. The decision support information may include recommendations for the worker to follow to avoid the alert situation.

The intervention/alert module 374 provides alerts based on system data. For example and not for limitation, alerts may be identified by the presence of data such as rapid HR acceleration, unnatural heart rate changes/variability, breathing cessation, motion cessation, atmospheric thresholds or sustained high core body temperature. The alert may further comprise descriptive interventions for the worker. The effect of these interventions/alerts is to effectively and efficiently communicate the urgency of the alert and improve the response time of the users while integrating the output of the decision support module 372.

The displays of the expert subsystem 370 may further communicate with a user interface (see 142 of FIG. 1B) to provide the user multiple views of integrated and contextualized data. Multiple displays may be arranged to provide an integrated overview of a geographical relationships and locations of multiple maintainers, a display with a high-level status overview of multiple maintainers, and a display with low-level details of a selected maintainer and the ambient environment.

### Monitoring Subsystem (CSS Server)—Interface

In one example embodiment, the CSS monitoring subsystem is a hypertext transfer protocol (HTTP) server that performs the following functions: Hosts web services feedback; Allows clients to interact with the system (and data) via the web results application programming interface (API); Hosts static web content for CSS Web Applications; Interacts with a message broker to stream and route real-time data to/from various endpoints; Executes algorithms in real-time to derive new data features and alerts within the system; and Saves all data sent to the server from the BAS and/or web applications.

The CSS Server directly interfaces with other components through three mechanisms: ActiveMQ Message Broker, HTTP Web Service and Content, and PostgreSQL Database via Java Database Connectivity (JDBC). Each of these interfaces is described below.

ActiveMQ is an open source message broker that provides real-time messaging capability for the CSS. The server interacts with ActiveMQ by leveraging the Java Messages Service (JMS) API (using an implementation provided by an ActiveMQ library). Through this API, the server establishes a connection to ActiveMQ and creates producers/consumers to send/receive data to/from various topics and queues.

The server also interfaces with a PostgreSQL database via JDBC. Currently, the server utilizes the Java Persistence API (JPA) to accomplish this, which serves as a wrapper around the JDBC connection. Using JPA and JDBC provides an abstraction between the CSS Server and PostgreSQL; given this, very little direct interaction is necessary. The direct interface between these two components is accomplished by using a JDBC driver for PostgreSQL, which is openly/freely available for use.

The server's main source of data transfer is done through its interaction with ActiveMQ. ActiveMQ supports many different types of transport mechanisms, but the server specifically uses the OpenWire format natively provided by ActiveMQ, which is transmitted over Transmission Control Protocol (TCP). The server handles requests through an HTTP server, which also uses TCP for its transport mechanism. Finally, the specific JDBC driver for PostgreSQL discussed in the previous section communicates using TCP as well.

Messages streamed to/from the server through ActiveMQ are binary encoded via the OpenWire protocol; however, the body of all these messages are text-based JavaScript Object Notation (JSON) messages. Currently, all the HTTP requests and responses handled by the server are also JSON encoded messages. The structure for all these data are documented in Appendix B. As mentioned in the previous section, a PostgreSQL JDBC driver is used to interface with the database; this makes underlying binary format of this data transparent to the CSS Server.

When the server indirectly interacts with BAS devices through ActiveMQ, it expects each BAS to send period status updates every 5 seconds. These updates serve as a heartbeat to the system. Every 5 seconds, the server checks all BASs to see if they have responded in the last 15 seconds. If they have not, it deems the BASs “disconnected” and broadcasts this info to the system.

The server also imposes time restrictions when sending status updates to the BAS, which can happen when the BAS's (or assigned person's) state changes. Through this channel of communication, the server first makes the change locally, and then attempts to send the update to the BAS. If the BAS does not recognize the state change and respond correctly within 3 seconds, the server will cancel the request and rollback any changes it made locally. During this period of time, the server will prevent other state changes to the BAS/person from happening until the initial request is finished.

When the server receives input data, the entity sending the data (e.g., the BAS) timestamps the message with the local creation time. This creation timestamp is expected to be relative to the server's local timestamp to account for system clocks not being in sync with each other. If the creation timestamp is older than 3 seconds when received, the server will deem the message “expired” and will not use it for further processing or persist it to the database.

### Web-Based Applications

In some embodiments, the CSS is able to store and provide access to users over a data network such as the Internet.

Referring to FIG. 3C, the CSS may comprise an HTTP server 391 and a web application database 392. The HTTP server 391 hosts web services 395 and static web content 393 (i.e., HyperText Markup Language (HTML), JavaScript, Cascading Style Sheets (CSS), etc.) for web clients to interact with. The web services 395 provide a web API 397 for clients to make requests through (using standard HTTP verbs). The web services 395 may further comprise a set of RESTful services 397 that interact with entities managed by the server 391. The web services 395 may also include Remote Procedure Call (RPC) services 398 that can be used to change the state of the system or worker. The web application database 392 may include information unique to the web application 390 or is may mirror or serve as the database for the CSS.

The CSS server 391 may also provide access to a set of web applications 394 to allow users to interface with the CSS via a web browser. These applications 394 are hosted by the CSS server 391 and can be accessed through various Uniform Resource Locators (URLs). Although these applications are used independently, they all use the same core services to interact with the system.

### Processor Based Embodiments of the Contextualized Sensor System:

The presented solution improves processor-based systems that attempt to remotely monitor via computer-based technology, in particular monitoring of workers in unconventional postures and spaces. The addition of context to health status alerts allows for remote systems, or a human remote attendant, to use contextualized data for optimal decision making in times of stress. The solution also minimizes the number of false positive alerts seen in similar systems, which undoubtedly contribute to at least a minimal amount of alert fatigue.

As will be readily apparent to those skilled in the art, contextualized sensor systems and methods of using them can be embodied in hardware, software, or a combination of hardware and software. For example, a computer system or server system, or other computer implemented apparatus combining hardware and software adapted for carrying out the methods described herein, may be suitable. One embodiment of a combination of hardware and software could be a computer system with a computer program that, when loaded and executed, carries out the respective methods described herein. In some embodiments, a specific use computer, containing specialized hardware or computer programming for carrying out one or more of the instructions of the computer program, may be utilized. In some embodiments, the computer system may comprise a device such as, but not limited to a digital phone, cellular phone, laptop computer, desktop computer, digital assistant, server or server/client system.

Computer program, software program, program, software or program code in the present context mean any expression, in any language, code or notation, of a set of instructions readable by a processor or computer system, intended to cause a system having an information processing capability to perform a particular function or bring about a certain result either directly or after either or both of the following: (a) conversion to another language, code or notation; and (b) reproduction in a different material form. A computer program can be written in any form of programming language, including compiled or interpreted languages, and it can be deployed in any form, including as a stand-alone program or as a module, component, subroutine, or other unit suitable for use in a computing environment.

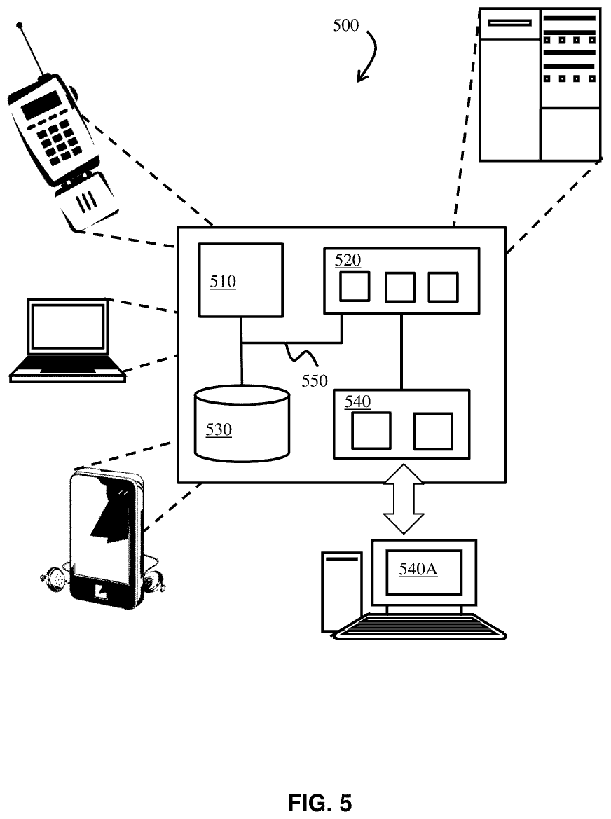

FIG. 5 is a schematic diagram of one embodiment of a computer system 500 by which the environmental system reaction methods may be carried out. The computer system 500 can be used for the operations described in association with any of the computer implemented methods described herein. The computer system 500 includes at least one processor 510, a memory 520 and an input/output device 540. Each of the components 510, 520, and 540 are operably coupled or interconnected using a system bus 550. The computer system 500 may further comprise a storage device 530 operably coupled or interconnected with the system bus 550.

The processor 510 is capable of receiving the instructions and/or data and processing the instructions of a computer program for execution within the computer system 500. In some embodiments, the processor 510 is a single-threaded processor. In some embodiments, the processor 510 is a multi-threaded processor. The processor 510 is capable of processing instructions of a computer stored in the memory 520 or on the storage device 530 to communicate information to the input/output device 540. Suitable processors for the execution of the computer program instruction include, by way of example, both general and special purpose microprocessors, and a sole processor or one of multiple processors of any kind of computer.

The memory 520 stores information within the computer system 500. Memory 520 may comprise a magnetic disk such as an internal hard disk or removable disk; a magneto-optical disk; an optical disk; or a semiconductor memory device such as PROM, EPROM, EEPROM or a flash memory device. In some embodiments, the memory 520 comprises a transitory or non-transitory computer readable medium. In some embodiments, the memory 520 is a volatile memory unit. In another embodiment, the memory 520 is a non-volatile memory unit.

The processor 510 and the memory 520 can be supplemented by, or incorporated in, ASICs (application-specific integrated circuits).

The storage device 530 may be capable of providing mass storage for the system 500. In various embodiments, the storage device 530 may be, for example only and not for limitation, a computer readable medium such as a floppy disk, a hard disk, an optical disk, a tape device, CD-ROM and DVD-ROM disks, alone or with a device to read the computer readable medium, or any other means known to the skilled artisan for providing the computer program to the computer system for execution thereby. In some embodiments, the storage device 530 comprises a transitory or non-transitory computer readable medium.

In some embodiments, the memory 520 and/or the storage device 530 may be located on a remote system such as a server system, coupled to the processor 510 via a network interface, such as an Ethernet interface.

The input/output device 540 provides input/output operations for the system 500 and may be in communication with a user interface 540A as shown. In one embodiment, the input/output device 540 includes a keyboard and/or pointing device. In some embodiments, the input/output device 540 includes a display unit for displaying graphical user interfaces or the input/output device 540 may comprise a touchscreen. In some embodiments, the user interface 540A comprises devices such as, but not limited to a keyboard, pointing device, display device or a touchscreen that provides a user with the ability to communicate with the input/output device 540.

The computer system 500 can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN, a WAN, wireless phone networks and the computers and networks forming the Internet.

One example embodiment of the contextualized sensor systems and methods of use may be embodied in a computer program product, the computer program product comprising a computer readable medium having a computer readable program code tangibly embodied therewith, the computer program code configured to implement the methods described herein, and which, when loaded in a computer system comprising a processor, is able to carry out these methods.

### One Embodiment of Methods to Contextualize Sensor System Data:

For illustration purposes and not for limitation, one example embodiment of the present invention is shown.

For illustration purposes and not for limitation, one example embodiment of the present invention is shown in FIG. 1A. As shown in FIG. 1A, the methods generally comprise receiving sensor data from one or more sensors one or more sensor, fusing the data to provide some contextualized data, comparing the contextualized data to a library of alert rules to determine whether an alert situation has occurred and if the alert situation has occurred, communicating an alert through a user interface. As shown, the sensors may comprise an environmental sensor, a location sensor, a physiological sensor, a behavior sensor and a posture sensor, and the sensor data may comprise an environmental data, a location data, a physiological data, a behavior data and a posture data.

In some embodiments, the contextual sensor system collects physiological data to also help assess cognitive readiness of the worker.

### Example Embodiments of the Contextualized Sensor System in Operation:

Operation of one embodiment of the contextualized sensor system is described below by outlining the steps, methods, and components that are required from process origin to completion. To assist with the description, please reference FIG. 1A and its associated functional components and descriptions.

The first step requires the instrumentation of the human and the environment. The human to be monitored will be equipped with the following sensors and devices: (1) a sensor-embedded garment or strap for cardiopulmonary monitoring, (2) a wearable or hand-held atmospheric monitor, which is equipped to sense pre-identified gas concentration (e.g., oxygen or volatile organic compounds), (3) a personal location tracking sensor unit or “puck”, (4) a smartwatch, and (5) a smartphone, though this device typically only need to be placed in the general vicinity of the human.

The environment also needs instrumented with location beacons (e.g., BLE- or UWB-enabled) to enable real-time location tracking in GPS-denied environments.

After the human operator has been equipped, the sensors will be turned on to ensure that the requisite data for optimal system utility is available. These data sources will then be fused to a centralized unit, which is colloquially referred to as a body area or personal area subsystem (BAS or PAS). The BAS for the presented application is currently a rugged smartphone due to their optimal processing power and various innate methods for wireless communication. While a smartphone currently does a satisfactory job of serving as a BAS, the system in operation could use other devices (e.g., LTE-enabled smartwatch), especially if the wearable technology industry and internet of things (IoT) initiative continues to progress. The five arrows in FIG. 1B show the transmission of data packets via BLE to the BAS, which is represented by the first black circle.

Though some onboard processing occurs (see “Pre-Processing & Transmission” in FIG. 1A) within the aforementioned sensors and components, there is a fair amount of data processing that still must occur. As such, the data is then transmitted via a major cellular infrastructure (e.g., Verizon Wireless LTE) to a cloud-based web server (e.g., Amazon Web Services [AWS]).

The next steps outlined in the process and corresponding figure (i.e., “Processed Real-Time Signals”, “Data Fusion for Contextualized Data”, and “Health & Safety Hierarchical Alerting Library”) all occur within the cloud-based web server.

First, the data are partitioned into 1-second queues. This step ensures that all data sources are temporally aligned—since many of the devices and subsequent data streams have disparate sampling rates.

The data streams are then used in isolation and concomitantly to provide context about the situation of the human in applied setting. A good example of this is the fusion of heart rate and accelerometer data. A higher-than-normal heart rate (e.g., 170 beats per minute [bpm] compared to a resting baseline of 70 bpm) can really only be labeled problematic if the observer has additional context about the situation. For example, this heart rate would be normal for an individual undergoing cardiovascular exercise. However, this heart rate would be abnormal for an individual doing work activities that require little or no strenuous exercise (e.g., putting in rivets in a supine position). This is where behavioral, location, and postural data can provide additional context.

The fusion of behavioral, location, and postural data sources can generally provide derived estimates of human activity through limited data analysis. For example, someone in an upright position, moving at a speed of about 1-1.5 m/s, and outside of a confined space, is likely walking based on a general understanding of the situation and normal human anatomy and physiology. In this instance, a slightly elevated heart rate is to be expected. However, if the data sources and associate analysis indicate that someone is laying on their side (i.e., recovery position) or front (i.e., prone), displaying little or no movement, inside of confined space, but presenting significantly elevated heart rate, there is likely an issue with the individual or the physiological monitoring equipment. Both of these contrived conditions would necessitate a prompt response from the local support staff. These instances provide an example of the “Alert Determination” section of FIG. 1A, where the data are fused with a pre-existing health and safety alerting architecture or library.

The “Health & Safety Hierarchical Alerting Library” houses pre-defined rules for health and safety alert generation. Most of these rules are based on combination of expected values (e.g., baselines), past data, normal physiology and behavior, and context. Some of the specific alerts housed in this library include: (1) sensor and network disconnects, (2) anomalous heart rate values and patterns, (3) anomalous respiration patterns, (4) detection of breathing cessation, (5) anomalous core body temperatures (both high and low), (6) anomalous atmospheric composition, and (7) low battery warnings. While non-exhaustive, this list provides a snapshot of the current alerting paradigm. Furthermore, it should also elucidate the relationship between raw sensor data and data context to allow for optimized alerting.

Lastly, the contextualized data and subsequent alerts then need to be communicated to the appropriate personnel. For the presented solution, these alerts—which are customized by job function—are transferred via user interfaces (UIs) to the human in the unconventional space or posture via smartwatch, to a roving (i.e., ground-based) attendant via tablet, and to a central monitoring station via desktop. While the specific methods for alerting may fall outside of the scope of this disclosure, the mechanism does not. These contextualize alerts can be fed to BLE- or internet-enabled hardware devices, such as tablets, smartwatches, or desktop computers.

In summary, the claimed invention includes a fusion of multiple data sources with contextual information to optimize the accuracy of alerts, which will ultimately be displayed on a hardware device. In other words, the fusion of data and context allows for more accurate and detailed information as to whether a dangerous incident is likely to ensue or currently underway.

This claimed innovation currently resides in a confined space monitoring system, though the fusion of physiological data, environmental data, location data, behavioral data, and postural data to provide context-enabled health and safety alerting could be applicable to various human monitoring applications.

### Example Application: Automated Maintainer Health Status Classification

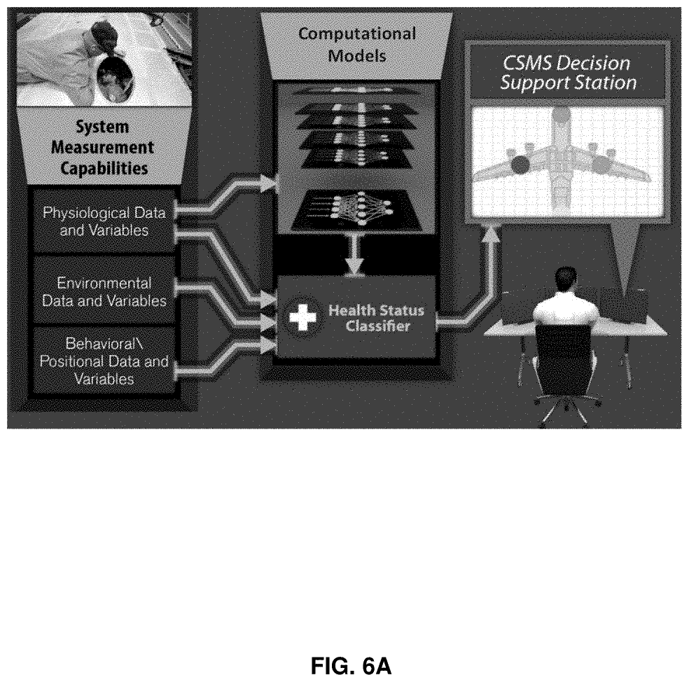

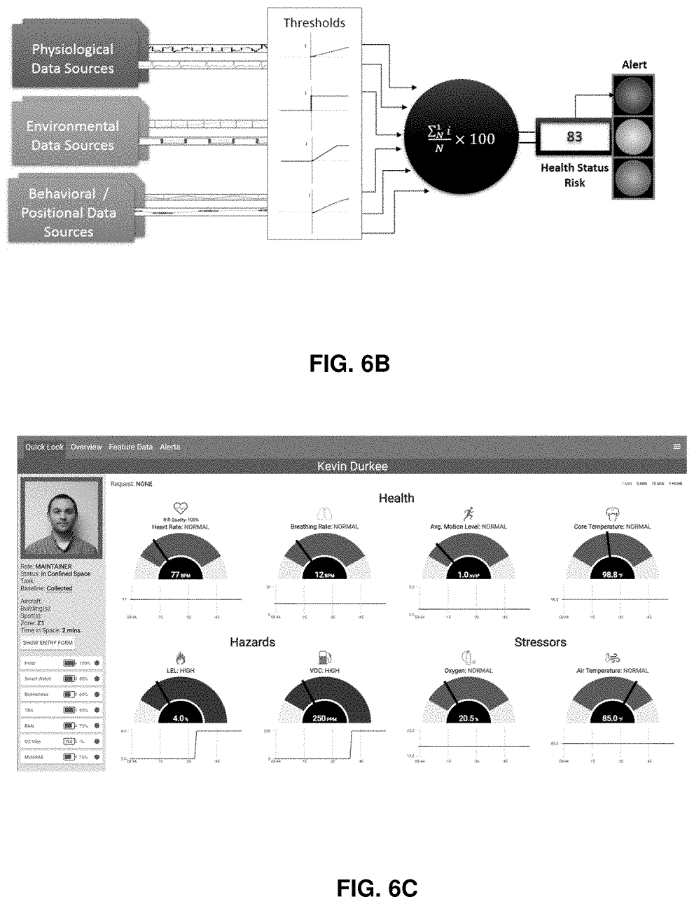

An automated, real-time health status assessment capability for each maintainer utilize a set of model-based classifiers that derive an estimate of maintainer health status by processing the various data sources collected by CSS (see FIG. 6A). Data inputs to a health status classifier include: physiological signals (heart rate, respiration); atmospheric levels (O₂, LEL, VOC); behavioral indicators (e.g., degree of physical movement); and physical location (e.g., inside a confined space).

The output of the health status classifier is a discrete state indicating one of three possible states a maintainer is in: optimal, sub-optimal, and emergency. An “optimal” state indicates that the subject is in no foreseeable risk of suffering an adverse outcome, and the majority of data sources are within desirable levels and not trending toward undesirable levels. A “sub-optimal” state indicates that one or more data sources are trending toward undesirable levels. Although a sub-optimal state does not constitute an emergency situation, the affected maintainer should be monitored more closely, and preventative intervention may be needed to ensure the maintainer's health/safety does not further degrade. An “emergency” state indicates that one or more data sources have exceeded desired levels, and that immediate intervention is required and justified, such as immediately vacating the affected maintainer from a confined space and, if needed, contacting EMS. As shown in FIG. 6A, these three possible health states are continuously fed to the decision support station. They are represented with a “stoplight chart” for each maintainer, in which an optimal state is green, sub-optimal state is yellow, and emergency state is red. The automated health status classifier provides the amount of time a maintainer has been within each state, since this information is critical to determining if and when an intervention is needed.

Two additional states were identified that are not specifically health related, yet remain important to properly prioritize the state of a maintainer. One is a disconnection status—this can refer to either a sensor disconnection (e.g., out of range, out of battery) or BAS disconnection from the CSS server (e.g., application crashes, Android device shuts off). In both cases, the same end result ensues in that the affected maintainer cannot be safely monitored remotely. Therefore, rapidly identifying these cases is of utmost importance. The maintainer disconnect status is color-coded as an “orange” state. The other case is that of a non-urgent (not safety critical) request for service, which is color-coded as a “blue” state. Service requests are typically (though not exclusively) initiated manually in situations when a maintainer needs help and it is not a matter implicating health and safety, such as needing a tool retrieved, requiring information, and receiving approval to enter a confined space.

Three distinct data processing layers were developed to facilitate the automated health status classification requirement for CSS: (1) signal processing; (2) data fusion; and (3) expert system. Signal processing refers to the conversion of raw signals into usable data features; it is a general term that can refer to various functions such as filtering noise, managing data volume, and refining data features before they are used in other system layers. The majority of signal processing in the CSS prototype is implemented on-board the wearable sensors and by BAS software on the Android device. Data fusion refers to the integration of multiple disparate measures to improve assessment capabilities, thereby better perceiving the “complete picture.” The expert system refers to alerting decision rules that flag potential existence of health/safety problems, while balancing with false alert tradeoffs. The majority of data fusion and expert system functions occur on the CSS server.

The expert system module comprises the situation classifier algorithms and table that monitor and apply each maintainer's data to classify health status as one of the three discretized states (optimal, sub-optimal, and emergency). As shown in FIG. 6B, the health status classifier defines thresholds and boundaries that are identified as sub-optimal, and with a certain level of severity, by monitoring the incoming data sources with respect to predefined values, thresholds, and ranges of values. Data sources are continuously monitored across the available categories of data (physiological, environmental, behavioral). An initial set of acceptable values, thresholds, and ranges for each variable was defined with a qualified multi-disciplinary team of healthcare experts, biomedical engineers, and human factors/safety specialists, to name a few. These parameters were selected with the expressed intention to maximize opportunities to detect health and safety incidents, while minimizing the likelihood of false alerts. Subsequently, the expert system underwent iterative modification throughout the project and, by the conclusion of the RIF effort, demonstrated the ability to effectively balance the need to alert legitimate issues (e.g., no-motion detected, excessively high heart rate over long durations, breathing cessation) while encountering a low false alert rate.

As part of this objective, an automated physiological baseline collection capability was implemented. This runs as an autonomous background service that examines recent windows of data to find sustained lower values using a Savitzky—Golay filter. Currently this processing routine is applied to heart rate data only, but it can be applied to any data feature as deemed necessary. The purpose in collecting baseline data is to allow a more individualized means for evaluating a person's data—i.e., relative to their baseline state. More specifically, the system computes “difference from baseline” metrics that can be used as the basis for health status classification and alerting. It is important to note the baseline capabilities assume an individual is healthy prior to entering a confined space.

Importantly, the CSS Gen1 sensor suite had to be defined and implemented to interoperate effectively with the maintainer health classifiers. Although the atmospheric sensor was essentially pre-selected to be the RAE Systems MultiRAE (O₂, LEL, VOC detection) based on prior LM-Aero experience and limited COTS alternatives being available, the COTS physiological sensor options were far more expansive. There were two primary selection criteria:

The physiological sensor suite must provide adequate quality of data to enable early warning and emergency detection of hazardous maintainer health states.

The sensors must offer a form factor that is comfortable, does not interfere with maintainer work, carries acceptable cost, and is sufficiently user-friendly to use.

To make a final decision, the design team relied on information and objective data provided by the AFRL 711^{th} Human Performance Wing, which has extensively tested a wide range of cardiopulmonary wearable sensors. Although some sensors are comparable in terms of cost and performance, the final down selections were made by allowing AMXG maintainers to view and try on inert sensors while crawling into non-permit confined spaces. This exercise led to the clear decision of using the Polar Team Pro electrocardiogram (EKG) base layer shirt, Polar H10 health sensor, and Samsung Galaxy Watch. Cardiopulmonary signals can be acquired by the Polar H10 when inserted into the Polar Team Pro shirt, while motion data can be acquired from the Galaxy Watch. As shown in the image below, the Galaxy Watch responded to Safety requests and end user preferences by mounting the watch unit into an arm band form factor. Of note, the Zephyr Bioharness remains available and fully compatible with this sensor suite (i.e., worn over the Polar Team Pro shirt for more robust respiratory data), but did not meet end user preferences due to comfort level and obtrusiveness.

### Example Application: Decision Support Station with Reliable Detection and Low False Alarm Rate

Technical objective #3 was to design and implement a decision support station that provides reliable detection of problematic events with a low false alert rate. The CSS use case requires moving away from the current 1-to-1 ratio of standby attendants per every person in a confined space, which will improve manpower utilization for Air Force sustainment, resulting in significant cost savings and accelerated maintenance schedules. However, employing a “many-to-one” ratio of confined space monitoring requires a new role in aircraft maintenance operations that is dedicated exclusively to monitoring maintainer health and safety. This role—referred to as the RSA—will ultimately be the main user of CSS.

A CSS decision support station must support the RSA's ability to coherently understand maintainer health and safety indicators, determine if and when a serious situation has (or is about to) occur, and initiate a timely intervention that is appropriate for the situation. To this end, the decision support station conveys four categories of real-time sensor data: physiological indicators; atmospheric hazards detected in the environment; maintainer locations; and behavioral activity detected by worn accelerometers. The overall health of each maintainer must also be clearly available from the health classifier algorithms (technical objective #2).

The decision support station includes a collection of informational displays and UIs spread across multiple computer screens, with the recommended layout being three computer monitors. Since the RSA role is new to the ALC confined space monitoring practices, the design team addressed this uncertainty through principled UI design with a human factors analysis and design approach that defines those CSS requirements in a way that matches how one would gather situation awareness and make the right decision at the right time.

The decision support station design includes a map-based display to intuitively communicate maintainer locations within a single view. Visual overlays (e.g., shape, color) are used to convey metadata for each maintainer. The UI is sufficiently flexible to scale up to a large number of maintainers to accommodate the size of ALC operations. Due to the large number of maintainers to monitor by a single RSA, the station design incorporates system alerting logic and “tripwires” to ensure potential issues are highly salient to RSAs. For example, if a maintainer's breathing rate drops below a certain rate per minute, the RSA will be alerted to this promptly. Alerts are delivered to the station both visually and through the use of auditory cues to reinforce these alerts. The ability to combine these system features under the decision support station ensures reliable detection is always present.

Stakeholder feedback also indicated a requirement to maintain a low false alert rate. Although occasional false alarms are expected and impossible to prevent altogether, a low false alert rate through the health status classifier and expert system design may be achieved by introducing a multi-modal sensor suite and cross-sensor data fusion to diversify how alerting decisions are made. More specifically, this is accomplished by the fact that if one sensor type fails or encounters an anomaly resulting in a false alert, the other sensors and data sources are present to prevent the false alert. For example, if breathing rate is suddenly not detectable for a maintainer due to a sensor malfunction, the presence of other sensor data such as heart rate, movement, location, and atmospheric levels provide a clearer picture of the cause. The automated data fusion provided by the health status classifier greatly helps with this interpretation of the data. Fast and easy recovery from false alerts is also key, which is accomplished through the station's UI design. The primary alerts information and resolution functionality is available to the RSA through the Primary Overview page. This UI also includes additional utility by automatically organizing all active maintainers according to their active work area and by their confined space entry status (i.e., not approved to enter, approved to enter, in confined space).

A maintainer status UI, FIG. 6C, was designed specifically to answer these questions in a fast and user-friendly manner, effectively using a dial graph for each question. Abnormal states are highly salient and alerts are readily viewable when they occur. Furthermore, the aforementioned maintainer disconnections (i.e., orange alerts) and service requests (i.e., blue alerts) are also rapidly identified through this UI. If an even more detailed look at the data is desired, the status UI includes an additional tab labeled “Feature Data” in which any data feature available for the selected maintainer can be viewed at any time and at any time scale (up to the past one hour). Further yet, data that spans beyond one hour in the past can be exported through a web-based data export tool and easily filtered and plotted through a third-party application (e.g., Excel).

Of note, the MultiRAE device runs on a proprietary RF channel implemented by RAE Systems that transmits solely to a ProRAE Guardian software application. In a notional ALC implementation, there would be a ProRAE Guardian application running on a machine in the ALC. This machine would run an application that connects locally to the ProRAE Guardian's API and transmits atmospheric sensor data directly to the CSS server using TCP/IP. A goal is to eventually replace the MultiRAE sensor with a smaller, more portable, and lower cost BLE-enabled atmospheric sensor.

The final aspect of the decision support station involves the electronic entry request sub-system. This sub-system ensures that maintainers are not allowed to enter a confined space until the RSA has acknowledged their request to enter. This is intended to ensure the maintainer's sensor data is visible and fully functional prior to entry, as well as to ensure the RSA is provided situational awareness regarding each maintainer's purpose for entry, time of entry, and approximate location. Each maintainer initiates this process by first entering atmospheric samples into a web-based UI for all confined spaces to be entered. This is followed by completing and submitting a confined space entry form. The entry form and corresponding atmospheric samples for the applicable confined spaces are made available to the RSA for review, at which point the RSA can approve the request to enter.

### Example Application: Intervention Support for Ensured Maintainer Health and Safety

Technical objective #4 was to produce system capabilities that support interventions to ensure maintainer health/safety through early detection and coordination with EMS teams. These capabilities are desired to increase the likelihood of preventing serious problems, and to accelerate response times when an intervention is needed. Although emergency response protocols are thoroughly planned by Safety personnel, the introduction of CSS brings an entirely new dimension to safety. CSS provides tools, information, and functionality that not only help fulfill the intent of current-day Safety protocols, but go a step further by leveraging the system capabilities for early interventions that serve as preventative measures to reduce the progression to more serious incidents.

To determine if and when a particular intervention is needed, CSS relies on health status classifiers and the decision support station to help the RSA decide if/when a maintainer has reached or is trending toward an undesired state. A “sub-optimal” (i.e., yellow) classification indicates that risk has elevated and there may be a need for preventative intervention in order to revert back to an “optimal” (i.e., green) state. Preventative interventions are designated for instances where a direct line of communication may need to be established with the maintainer to advise suggested actions, but there are no current emergency situations nor any need for coordination with EMS. Examples of lower risk events that could require a preventative intervention are: rising heat stress levels; small but rising remnants of flammable or volatile hazards detected by the atmospheric sensor; and minor physiological irregularities such as slightly higher/lower than usual heart rate or breathing rate. Going beyond mere preventative interventions, the detection of a more serious and/or time-sensitive situation is classified as an “emergency” (i.e., red) state, indicating that risk is beyond an acceptable threshold and immediate action should be taken (at a minimum, the maintainer should be removed from a confined space). Examples of higher risk and more time-sensitive events that require immediate intervention are: extreme heat stress levels during heat advisory weather conditions; rising levels of flammable or volatile hazards detected by the atmospheric sensor; and dangerous physiological symptoms such as excessively high/low heart rate while in a stationary position (e.g., 140 heartbeats per minute).

Based on health classifier assessments, the intervention response is then determined by the RSA, in many cases with the aid of CSS. As stated by Galster and Johnson (2013) in AFRL's Sense-Assess-Augment framework, augmentation is often very context-specific and may require a unique intervention depending on the specific person, event, time, and location. The confined space monitoring use case is no exception to this rule. This requires the ability to define, communicate, and execute uniquely crafted COAs that fit the specific need. The concept of COAs is derived from the Military Decision Making Process (MDMP; Army FM 5-0), and has been successfully adapted to other domains including Air Force emergency response (e.g., Air Force Emergency Operations Centers, or EOCs). To ensure the right interventions are delivered for a given situation, collaborations were made with maintenance personnel at LM-Aero's C-5 complex and WR-ALC to identify the most dangerous and most likely health and safety events. Based on these events, the team coordinated with Safety to formulate approved intervention strategies in current health/safety response protocols, whether preventative in nature or emergency response oriented.

For many cases, particularly emergency situations, there may be a common and straightforward COA for a RSA to direct a maintainer to remove himself/herself from a confined space; or in more extreme cases, to call 911 to notify the EMS or fire department responsible for a given location. However, in more ambiguous and less time-sensitive circumstances, such as “sub-optimal” status indications described previously, the RSA may find it challenging to know all possible responses, particularly given the various possible health/safety hazards that could exist and their low frequency of occurrence. For this reason, the design team formulated a standardized ConOps for CSS that employs a specific set of responses by system user depending on the color-coded alert. Specifically, there is one COA defined per each alert level: blue, yellow, orange, and red. In virtually all cases, the RA's role is essential as the “eyes and ears” of the RSA on the production floor, and therefore is intended to be the first person to initially investigate the source of an alert, regardless of priority level. The CSS ConOps intends the RSA and RA to maintain continuous verbal communication via hand radios so that any alerts that occur are clearly and concisely communicated before taking the remedial action. Below is a high-level description for each response according to the alert level.

Blue alert: Indicates that non-urgent help is requested by maintainer (typically manually initiated by maintainer), and that the issue does not implicate the maintainer's health and/or safety in any way. The RSA and RA provide a response as able, provided there are no higher severity alerts.

Yellow alert: Indicates the health status classifier detects a sub-optimal state for a specific maintainer, but has not yet reached emergency levels that would merit contacting EMS. The purpose of this alert is to serve as an early warning. The RSA's and RA's goal is to provide immediate response in case the issue is trending toward a red state. If another red alert exists at this time, maintainer in yellow state must immediately evacuate the confined space.

Orange alert: Indicates the maintainer has a disconnection with their associated sensor kit that is blocking the desired level of remote health/safety monitoring. This issue may occur when the sensor battery level is low, the sensor is out of range, the mobile device powers off, or the BAS application crashes. The RSA's and RA's goal is to provide immediate response in case the issue is caused by an unsafe event and/or in case an incident occurs that cannot be detected due to the disconnection. If another yellow or red alert exists at this time, maintainer in orange state must evacuate the confined space; otherwise, the RA's goal is to assist with resolving the issue.

Red alert: Indicates the health status classifier detects an emergency level state for a specific maintainer. The RSA and RA must respond with full haste to confirm that an emergency has indeed occurred. The RA's goal is to reach the confined space entry hatch within 60 seconds and verify the issue. If a false alert occurs, the RA verbally communicates this to RSA so the alert is promptly resolved and marked accordingly from the decision support station. Additionally, an optional feature to minimize response time is that EMS personnel (e.g., Fire Dept) are automatically notified of the red alert (if provided a CSS Geoview page at the EMS terminal station), so the exact location of the affected individual is provided. If the EMS does not receive a false alert response within a certain amount of time (e.g., 90 seconds), they may automatically deploy to the scene. This ConOps decision shall be made by the applicable government Safety personnel.

Additionally, the RSA's decision support station includes a special system feature in which the RSA can initiate an “evacuate all” command to any maintainers working in a specific zone. In practice this is most similar to a red alert in that the primary goal is to immediately cease work activities and vacate the confined space. However, the evacuation command is unique in that it is initiated by the RSA, rather than being automated, and is intended to affect everyone working in confined spaces at a given time. The reason to issue evacuation commands can range from the presence of a volatile hazard that has entered a particular building (and thus affecting multiple confined spaces in that building) to the EMS or Fire Department teams being occupied and unavailable at a particular time.

The CSS ConOps and COA intervention strategies were coordinated with Safety personnel representing all three ALCs (Warner Robins, Tinker, and Hill) throughout the project in the form of a Safety Integrated Product Team (IPT). The goal was to ensure 100% compliance with mandatory preventative measures and emergency response protocols, as well as to identify protocols and documentation that may need to be revised based on the introduction of CSS. This was to ensure there is preemptive acceptance by Air Force Safety for using CSS to support the identification, prevention, and resolution of health and safety issues, as well as to ensure there are no violations that would lead to wasted time and resources to resolve during the eventual technology transition phase.

To execute this ConOps and ensure the effective delivery of preventative health and safety interventions, it was also very important to consider the most effective delivery medium to communicate a COA directly to both the RAs and the affected maintainer. In current depot operations, verbal radio communication is typically the single exclusive communication medium relied upon to provide directions. Although this has proven to be effective, it can often be slow and inefficient from the perspective of optimizing time, manpower, and attention, particularly for a single RSA responsible for monitoring dozens of other maintainers. For this reason, a part of CSS technical approach was to incorporate non-verbal methods of communication and information exchange through human-wearable technologies.

COTS wrist-worn devices, or “smart watches,” were extensively reviewed and tested for non-verbal communication to ensure they would fit well into the CSS concept. Wrist-worn devices were explored based on recommendations by LM-Aero's C-5 maintenance personnel given their experience with the remote monitoring concept. This was further enabled thanks to significant technological advancements over the past several years in the smartwatch industry. Furthermore, the ability to converge multiple CSS functions (i.e., motion sensors and maintainer UI) into a single non-invasive wearable device would greatly improve user reception and overall effectiveness of the system. Although a smartwatch does not replace or adequately substitute verbal radio communication for many situations, under the right circumstances—such as requests to enter a confined space, requests for help, mass notifications (e.g., evacuate confined spaces), and basic acknowledgements—a non-verbal indicator sent via wrist-worn system interactions provides a faster and less costly means to track maintainer status information and convey information without affecting maintainer health/safety. Of note, maintainers requested an arm band form factor to holster the smartwatch to avoid having to wear on a wrist.

The Samsung Galaxy Watch is an example means to unobtrusively measure continuous motion level on a maintainer through a work shift. The added benefit in using this device is its wide set of capabilities that can fulfill several intervention requirements. Specifically, the Galaxy Watch can run software that performs virtually all of these functions, including: communicating your confined space entry status (e.g., pending approval to enter, approved to enter, inside space); calling for a service request (blue alert); calling for critical help (red alert); and receiving notifications when an alert has occurred. This Galaxy Watch application also provides a way to display a special form of alert—deemed an Aqua alert—that communicates directly to the maintainer that a pending alert is getting ready to trigger. The Aqua alert occurs prior to reaching the RSA decision support station so benign situations (e.g., no-motion alerts caused by sitting still for too long) are identified before causing a false alert situation FIG. 4C illustrates the worker UI built for the Galaxy Watch with the arm band form factor.

Similar benefits to those provided for maintainer communication can be realized by providing the smartwatch application to RAs. Specifically, anytime a maintainer in the RA's zone triggers an alert or call for help, the RA receives this alert on their smartwatch UI. RSA's Geoview can be built specifically for RA usage on a mobile device. This UI displays the RA's current location in the center of the screen and upon selecting the maintainer with an alert status, a line is overlaid to connect the RA's location to that maintainer. The distance (number of feet) to reach the affected maintainer is also displayed.

To ensure RAs are not asked to cover an unreasonably sized area—an essential requirement to assure they can respond to alerts in the minimum amount of time needed—it is important to note the CSS ConOps also forces RAs to be assigned to a specific zone. If multiple zones are defined, then multiple RAs are needed to cover each zone at a one-to-one ratio. To support this requirement, all CSS Geoview displays provide the option to display zone overlays. Zone configurations are completely configurable by an administrator panel.

For emergency situations that require a time-sensitive response by an outside agency (e.g., local EMS or fire department), CSS provides a data publishing service that supplies critical information (e.g., alert states, maintainer locations, number of maintainers in confined spaces) directly to the first response team. Although distribution of this information to response teams is an optional feature, it can reduce coordination time between the RSA, RA, maintenance control, and emergency responders. The data publishing service is facilitated by the use of the CSS cloud-based services and the use of secure web technologies that can be easily accessed through conventional web browsers (e.g., Google Chrome). Each applicable organization has the option of using one of the existing read-only versions of the CSS displays, such as the Geoview.

Research Results from One Example Embodiment:

CSS is a sensors-based system for remote health and safety monitoring of maintainers working in confined spaces. CSS has two main aspects: an unobtrusive sensor suite worn by maintainers and an integrated decision support station for alerting and intervention. In the embodiment tested, the sensor suite collects real-time measurements of maintainers' health signals (heart rate, breathing, motion), atmospheric levels in their vicinity (oxygen, LEL, VOCs), and location in GPS-denied environments. A decision support station provides remote monitoring for a single safety attendant to safely monitor the health and safety of many confined spaces concurrently. This is designed especially for early-warning detection for preventative intervention and accelerating response by EMS personnel.

CSS utilizes four classes of technical components: portable sensors, data networking, remote monitoring displays, and alerting and intervention. In the embodiment tested, the CSS packages portable sensors into “sensor kits” assigned to a specific maintainer prior to entering a confined space. A sensor kit consists of health, atmospheric, and location sensors. The health sensors used with the current prototype are the Polar Team Pro base layer shirt, Polar H10 wireless unit, and Samsung Galaxy smartwatch with arm band holster. The Polar Team Pro and H10 unit collect real-time heart rate and R-R intervals from its user. The Samsung Galaxy smartwatch is used to measure real-time actigraphy (motion levels) from its user, while offering a communication display to receive alerts/notifications and calls for help as needed. The atmospheric sensor used with the current CSS prototype is RAE Systems' MultiRAE Pro, which measures atmospheric data from the maintainer's immediate surroundings. Location sensors are provided by TRX Systems' NEON indoor tracking system, a hybrid solution that uses BLE-based iBeacons, Ultra-Wideband beacons, MEMS-based inertial navigation, and additional constraints and pre-mappings to optimize accuracy.

The Gen1 data network consists of BLE short-range data communication, 4G LTE frequencies for long-range communication, and Amazon Web Services GovCloud for real-time processing and communication to the monitoring displays. BLE is used to connect each portable sensor to a mobile device running the BAS software program. The BAS mobile device manages the receipt, local processing, and relay of sensor data to the cloud server via a wireless network provider's 4G LTE (e.g., Verizon, AT&T). The cloud services manage real-time data processing, alert generation, data storage, and remote display access via a web server to approved clients on additional networks, such as Verizon MiFi Hotspots or AFNET.

This current CSS embodiment provides three primary remote safety monitoring displays: Geoview, Primary Overview, and Maintainer Status. Because these are web-based client applications, they are accessible from a wide range of devices, including desktop computers, tablets, and cell phones. The Geoview overlays maintainers' location data on a map display along with their current health status, allowing a prompt and accurate response to their location if a potential emergency occurs. The Personnel Overview display provides a card-based view of each maintainer checked into CSS at a given time, while illustrating their current health status and detailed information on any system-generated alerts. The Personnel Overview display is also the remote attendant's primary tool for navigating the overall remote monitoring station, offering features such as acknowledgement of confined space entries, maintainer selection, zone evacuation commands, alert management, and auto-sorting maintainers inside vs. outside confined spaces. The Status display allows viewing of a specific maintainer's sensor data at a greater level of detail than the previous displays. In particular, this display allows viewing of both current sensor readings and recent historical sensor data that may be relevant to the current health and safety status.

The alerting and intervention layer of CSS consists of a backend alert generation module that drives specific display behavior on the Geoview, Personnel Overview, and Status displays. In response, the associated ConOps dictates the system end users to follow a specific intervention protocol to rapidly confirm (or deny, in false alert instances) the existence of the detected event, followed by completion of all necessary steps to ensure the safety of the affected individual. The alert generation module follows a color-coded scheme tied to a specific intervention response by safety attendants. This current CSS embodiment is programmed to classify each maintainer as a Green state unless specific alerting criteria are met. The current embodiment contains a library of algorithms that each probe for a specific state, and if detected, the maintainer is classified into the new state accordingly. The algorithms library can be updated and expanded as further system testing is conducted and/or if new additional sensors are added to the maintainer sensor kits.

### CSS Alert Configurations:

Below are examples of CSS alerts as configured for a technical demonstration. Due to minimal training time available with AMXG maintainers, there was elevated likelihood of false positives. Due to this limitation, alerts were set with more conservative parameters to minimize interruptions to maintainer work.

1. Cardiopulmonary Alerts:

Adaptive HR (AHR) threshold is defined as either 144 bpm, or [HR Baseline 2.125], whichever is less. Since most maintainers will likely baseline >67.8 bpm due to heat and physical activity, it is likely that 144 bpm will be the AHR most, if not all, the time. (BL<67.8 bpm will cause AHR to incrementally drop.)

YELLOW—immediately triggers if Heart Rate reaches 180+ bpm.

YELLOW—triggers if Heart Rate goes above AHR for 2-minutes.

YELLOW—triggers if Core Body Temp algorithm exceeds 101.5 degrees Fahrenheit.

RED—triggers if Heart Rate is 180+ bpm for 30+ seconds.

RED—triggers if Heart Rate goes above AHR for 3-minutes.

RED—triggers if Core Body Temp algorithm exceeds 102.5 degrees Fahrenheit.

2. Motion Alerts. No-motion threshold is defined as average Motion Level being <0.08 on Smartwatch.

YELLOW—triggers if no motion detected for 3-min.

RED—triggers if no motion detected for 10-min.

3. Atmospheric Sensor Alerts:

YELLOW—Very close to reaching High/Low Oxygen levels (below 20%, above 23%).

YELLOW—Very close to reaching High LEL (above 5%).

YELLOW—Very close to reaching High VOC (above 300 ppm).

RED—detected High/Low Oxygen levels (below 19.5%, above 23.5%).

RED—detected High LEL (above 10%).

RED—detected High VOC (above 600 ppm).

4. Entrant-initiated Alerts:

BLUE—Requesting Approval to Enter: maintainer submitted entry request; RSA needs to press Approve before maintainer is allowed to signal entry into space.

BLUE—Service Request: maintainer indicated a non-safety critical call for help. RSA should press Acknowledge button on Status page and resolve once RA investigates.

RED—Call for Help: maintainer indicated a potentially safety-critical call for help. RSA should press Acknowledge button on Status page and resolve once RA investigates.

5. Maintainer-only Alerts:

AQUA—High HR pre-warning: immediately triggers if Heart Rate reaches 170+ bpm.

AQUA—Adaptive HR pre-warning: triggers if HR goes above AHR for 60-sec.

AQUA—High Core Body Temp: triggers if Core Body Temp exceeds 100.5 degrees Fahrenheit.

AQUA—No-motion pre-warning: triggers if no motion detected at 60-sec & 120-sec.

AQUA—LEL awareness: triggers if LEL is above 2%.

AQUA—VOC awareness: triggers if VOC is above 120 ppm.

AQUA—InSpace query: triggers a query if InSpace Detection algorithm thinks person is inside a confined space and maintainer has not yet pressed “Enter Space” button to signal entry. (Side Note: Algorithm is not sufficiently robust to automate the entry signal, so it prompts user to query instead, in case maintainer forgets to signal entry themselves.)

6. Connectivity & Battery Alerts:

YELLOW—Sensor/Smartwatch/BAS has low battery (<10% remaining). FIX: Battery should last for additional ˜30 minutes upon this warning occurring. Only immediate fix is to assign a new kit that is charged, then start re-charging the kit that has a low-battery component.

ORANGE—Sensor/Smartwatch disconnects from BAS—visible on Status page. FIX: Sensor/Smartwatch must be in range of BAS. If problem persists, ask system administrator.

ORANGE—BAS disconnects from server for 15+ sec—visible on person's Status page. FIX: May be in area with poor 4G LTE coverage. If Verizon, consider switching to different kit that has AT&T (or vice-versa). Also ensure phone battery life is adequate. If problem persists, inspect BAS app and/or re-start BAS.

ORANGE—RSA station disconnects from server—indicated by Orange pop-up message. FIX: Most likely issue is internet disruption or cloud server is down. Only fix is to resolve core web access and/or cloud server problem. If problem persists, ask system administrator.

Although this invention has been described in the above forms with a certain degree of particularity, it is understood that the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention which is defined in the claims and their equivalents.

1. A processor based contextualized sensor system comprising:
a body area subsystem comprising one or more sensors configured to collect state data of a worker;
a monitoring subsystem comprising:
an expert subsystem comprising a situation classifier module and an intervention/alert module,
a subsystem database comprising contextualization rules and a library of alert rules,
the situation classifier module is configured to determine a contextualized situation from one or more sensor data using the contextualization rules, and the intervention/alert module configured to compare the contextualized situation to the library of alert rules to determine whether an alert situation has occurred; and
if the alert situation has occurred, the monitoring subsystem configured to communicate an alert.

2. The processor based contextualized sensor system of claim 1 wherein the situation classifier module further comprises a state contextualizer module configured to determine a contextualized state of the worker from the one or more sensor data.

3. The processor based contextualized sensor system of claim 2 wherein the contextualized state is determined from a posture data.

4. The processor based contextualized sensor system of claim 2 wherein the contextualized state is determined from a location data.

5. The processor based contextualized sensor system of claim 2 wherein the contextualized state is determined from a comparison of the one or more sensor data to a baseline data of the one or more sensors.

6. The processor based contextualized sensor system of claim 1 wherein the one or more sensor data comprises a posture data.

7. The processor based contextualized sensor system of claim 1 wherein the one or more sensors comprise a posture sensor.

8. A processor based contextualized sensor system for determining a contextualized state of a worker, the system comprising:
a body area subsystem comprising one or more sensors configured to collect state data of a worker; and
a monitoring subsystem comprising:
an expert subsystem comprising a situation classifier module,
a subsystem database comprising contextualization rules,
the situation classifier module is configured to determine a contextualized state of the worker from the state data.

9. The processor based contextualized sensor system of claim 8 wherein:
the contextualization rules comprise a raw variable, a contextualization variable and a resulting contextualized variable;
the raw variable comprises a first raw state data of the worker;
the contextualization variable comprises a second raw state data of the worker; and
the resulting contextualized variable comprises an objective representation of the contextualized state of the worker given the first raw state data and the second raw state data of the worker.

10. (canceled)

11. The processor based contextualized sensor system of claim 9 wherein the second raw state data comprises a comparison metric of a core body temperature of the worker to a baseline core body temperature of the worker.

12. The processor based contextualized sensor system of claim 9 wherein:
the second raw state data comprises an orientation data of the worker; and
the orientation data of the worker comprises a posture data of the worker.

13. The processor based contextualized sensor system of claim 8 wherein:
the body area subsystem further comprises one or more environmental sensors configured to collect environmental data; and
the situation classifier module is further configured to determine a contextualized state of the worker from the state data and the environmental data.

14. The processor based contextualized sensor system of claim 13 wherein:
the contextualization rules comprise a raw variable, a contextualization variable and a resulting contextualized variable;
the raw variable comprises a raw state data of the worker;
the contextualization variable comprises an environmental data; and
the resulting contextualized variable comprises an objective representation of the contextualized state of the worker given the raw state data and the environmental data.

15. (canceled)

16. The processor based contextualized sensor system of claim 14 wherein the state data comprises a comparison metric of a core body temperature of the worker to a baseline core body temperature of the worker.

17. The processor based contextualized sensor system of claim 14 wherein the environmental data comprises a location data of the worker.

18. The processor based contextualized sensor system of claim 14 wherein the environmental data comprises a carbon monoxide level.

19. The processor based contextualized sensor system of claim 6 wherein the one or more posture data comprises one or more body posture metric derived from an orientation of an inertial measurement unit (IMU) compared to a pre-calibrated orientation baseline defining a posture of the worker as being in a prone posture or in a standing posture.

20. The processor based contextualized sensor system of claim 7 wherein the posture sensor comprises an inertial measurement unit (IMU) configured to provide posture data comprising a body posture metric derived from an orientation of the IMU compared to a pre-calibrated orientation baseline defining a posture of the worker as being in a prone posture or a standing posture.

21. The processor based contextualized sensor system of claim 1 wherein the one or more sensor comprises an accelerometer configured to provide a motion data of the worker.

22. The processor based contextualized sensor system of claim 8 wherein the one or more sensor comprises an accelerometer configured to provide a motion data of the worker.
